(19) United States
(12) Patent Application Publication
(10) Pub. No.: US 2024/0424286 A1
(43) Pub. Date: Dec. 26, 2024

(54) MINIMALLY INVASIVE MEDICAL DEVICE DELIVERY

(71) Applicant: EDWARDS LIFESCIENCES CORPORATION, Irvine, CA (US)

(72) Inventors: Hien T. Nguyen, Aliso Viejo, CA (US); Daniel James Murray, Orange, CA (US); Michael G. Valdez, Riverside, CA (US); Yair A. Neumann, Moshav Sede Varburg (IL); Anne Bernadette Aragon Alcasid, Costa Mesa, CA (US); Amir Davidesko, Binyamina Givat Ada (IL); Steven Park, Santa Ana, CA (US); Sofia Rozenberg, Tel-Aviv (IL)

(21) Appl. No.: 18/823,586

(22) Filed: Sep. 3, 2024

Related U.S. Application Data
(63) Continuation of application No. PCT/US2023/014466, filed on Mar. 3, 2023.
(60) Provisional application No. 63/317,498, filed on Mar. 7, 2022.

(51) Int. Cl. A61M 60/865; A61M 25/00; A61M 60/187; A61M 60/289; A61M 60/465

(52) U.S. Cl. CPC A61M 60/865; A61M 25/005; A61M 60/187; A61M 60/289; A61M 60/465

(57) ABSTRACT

A medical device delivery system can comprise a delivery catheter and an in-line sheath, the delivery catheter being preloaded within the in-line sheath. The delivery catheter can comprise a catheter hub and a catheter shaft. The catheter shaft can comprise a proximal portion extending distally from the hub and slidably receiving a proximal narrow portion of a medical device, and a distal portion slidably receiving a distal wide portion of a medical device. The in-line sheath can comprise a sheath hub and a sheath shaft. The sheath shaft can comprise a proximal portion extending distally from the sheath hub and slidably receiving at least a portion of the proximal portion of the catheter shaft, and an expandable distal portion slidably receiving the distal portion of the catheter shaft.

### CROSS REFERENCE TO RELATED APPLICATIONS

This application is a continuation of International Patent Application No. PCT/US2023/014466, filed Mar. 3, 2023, entitled “SYSTEMS FOR MINIMALLY INVASIVE DELIVERY OF MEDICAL DEVICES,” which claims the benefit of U.S. Provisional Patent Application No. 63/317,498, filed Mar. 7, 2022, entitled “MEDICAL DEVICE DELIVERY SYSTEM,” the disclosures of each of which are hereby expressly incorporated by reference herein in their entireties for all purposes.

### BACKGROUND

The present disclosure generally relates to the field of minimally invasive delivery of medical devices and/or therapies. Minimally invasive transcatheter delivery of medical devices and/or therapies to a target site within the body can be utilized to treat any number of conditions. Medical devices and/or therapies can be delivered into the heart to address various heart abnormalities.

### SUMMARY

Described herein are methods and devices relating to a medical device delivery system configured to allow advancement of a medical device through the delivery system for minimally invasive transcatheter delivery of the medical device. The medical device delivery system can comprise an in-line sheath and a delivery catheter associated with the in-line sheath. For example, the delivery catheter can be preloaded within the in-line sheath to facilitate advancement of the medical device delivery system through tortuous anatomical pathways.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 is a cross-sectional view of a human heart.

FIGS. 2A, 2B and 2C are side cross-sectional views of an example of a medical device delivery system.

FIGS. 3A, 3B and 3C are side cross-sectional views of a sheath delivery system comprising the medical device delivery system described with reference to FIG. 2 positioned through an outer expandable sheath.

FIG. 4 is a perspective view of an example of a sheath shaft that includes an expandable distal portion comprising an expandable coil.

FIG. 5 is a perspective view of an example of a sheath shaft that includes an expandable distal portion comprising shape memory material.

FIG. 6 is a perspective view of an example of a sheath shaft that includes an expandable distal portion comprising a plurality of discrete elongate portions circumferentially disposed around a delivery catheter conduit extending through the sheath shaft.

FIG. 7A is a side view, and FIG. 7B is a side cross-sectional view, of an example of a sheath shaft that includes an expandable distal portion comprising a plurality of discrete elongate portions, each discrete elongate portion extending distally along an axis parallel or substantially parallel to a longitudinal axis of the sheath shaft.

FIG. 8A is a side view, and FIG. 8B is a side cross-sectional view, of an example of a sheath shaft that includes an expandable distal portion comprising a plurality of discrete elongate portions, each discrete elongate portion having a bend on a proximally oriented end portion.

FIG. 9A is a side view, and FIG. 9B is a side cross-sectional view, of an example of a sheath shaft that includes an expandable distal portion comprising a plurality of discrete elongate portions, each discrete elongate portion extending along a convexly curved path from a proximal end to a distal end.

FIG. 10A is a side view, and FIG. 10B is a side cross-sectional view, of an example of a sheath shaft that includes an expandable distal portion comprising a plurality of discrete elongate portions, each discrete elongate portion having a convex curvature on a proximally oriented end portion.

FIG. 11 is a side perspective view of a sheath shaft comprising three portions, each portion comprising a material different from the other portions.

FIG. 12 is a side cross-sectional view showing a distal end of an example of a sheath shaft, where the distal end can be configured to mate with a distal portion of a medical device received within the sheath shaft.

FIG. 13 is a side cross-sectional view showing a distal end of another example of a sheath shaft, where the distal end can be configured to mate with a distal portion of a medical device received within the sheath shaft.

FIG. 14 is a flow diagram of an example of a process for deploying a medical device using a medical device delivery system as described herein.

FIG. 15 is a flow diagram of an example of a process for manufacturing a medical device delivery system as described herein.

### DETAILED DESCRIPTION

The headings provided herein are for convenience only and do not necessarily affect the scope or meaning of the claimed invention.

The present disclosure provides systems, devices, and methods relating to a medical device delivery system which can be used to facilitate minimally invasive transcatheter delivery of a medical device to a target site. The medical device delivery system can comprise an in-line sheath and a delivery catheter preloaded within the in-line sheath. The medical device can comprise a proximal narrow portion and a distal wide portion, the distal wide portion comprising a larger lateral cross-sectional size than that of the proximal narrow portion. The medical device can be preloaded into the delivery catheter such that the proximal narrow portion is received within a proximal portion of a catheter shaft of the delivery catheter and the distal wide portion is received within a distal portion of the catheter shaft. The delivery catheter comprising the medical device preloaded therein can be preloaded within the in-line sheath such that the proximal portion of the catheter shaft is received within a proximal portion of a sheath shaft of the in-line sheath and the distal portion of the catheter shaft is received within an expandable distal portion of the sheath shaft. The medical device delivery system comprising the delivery catheter, with the medical device received therein, preload within the in-line sheath can facilitate advancement of the medical device delivery system through tortuous anatomical pathways.

Although certain preferred instances and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular embodiment. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

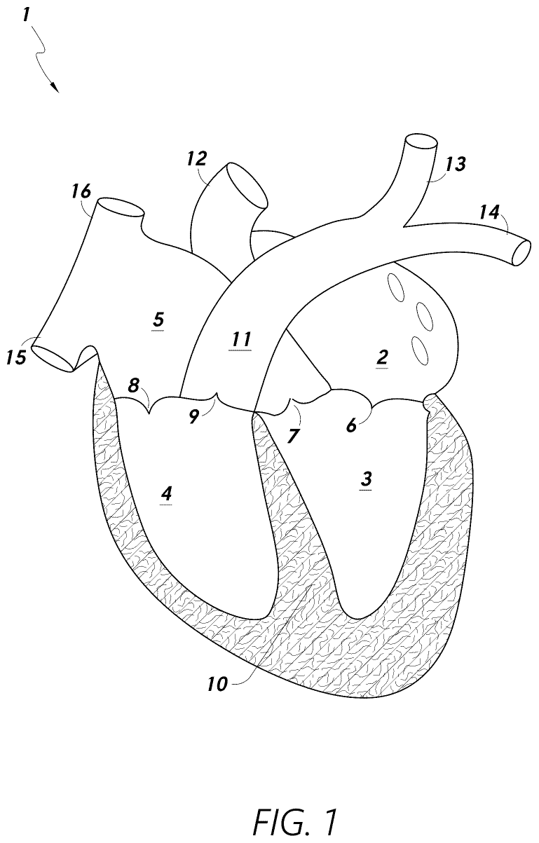

FIG. 1 is a schematic diagram showing various features of a human heart 1. The heart 1 includes four chambers, namely the left atrium 2, the left ventricle 3, the right ventricle 4, and the right atrium 5. A wall of muscle, referred to as the septum 10, separates the left atrium 2 and right atrium 5, and the left ventricle 3 and right ventricle 4. Blood flow through the heart 1 is at least partially controlled by four valves, the mitral valve 6, aortic valve 7, tricuspid valve 8, and pulmonary valve 9. The mitral valve 6 separates the left atrium 2 and the left ventricle 3 and controls blood flow therebetween. The aortic valve 7 separates and controls blood flow between the left ventricle 3 and the aorta 12. The tricuspid valve 8 separates the right atrium 5 and the right ventricle 4 and controls blood flow therebetween. The pulmonary valve 9 separates the right ventricle 4 and the pulmonary trunk or artery 11, controlling blood flow therebetween.

In a healthy heart, the heart can receive deoxygenated blood arriving from the rest of the body generally into the right side of the heart for transport to the lungs, and oxygenated blood from the lungs generally into the left side of the heart for transport to the rest of the body. During ventricular diastole, deoxygenated blood arrive in the right atrium 5 from the inferior vena cava 15 and superior vena cava 16 to flow into the right ventricle 4, and oxygenated blood arrive in the left atrium 2 from the pulmonary veins to flow into the left ventricle 3. During ventricular systole, deoxygenated blood from the right ventricle 4 can flow into the pulmonary trunk 11 for transport to the lungs (e.g., via the left 14 and right 13 pulmonary arteries), and oxygenated blood can flow from the left ventricle 3 to the aorta 12 for transport to the rest of the body.

A number of conditions can inhibit the ability of the heart 1 to transport sufficient blood therethrough. For example, coronary heart disease, heart inflammation, and/or high blood pressure can contribute reduced blood flow through the heart 1. Dysfunction of the mitral valve can contribute to elevated left atrial pressure. Conditions such as mitral valve regurgitation and/or stenosis may result in difficulty in pumping blood from the left atrium to the left ventricle, contributing to elevated pressure in the left atrium. Dysfunction in the left ventricle 3 can also contribute to elevated left atrial pressure.

The disclosure herein provides one or more devices and methods related a medical device delivery system comprising an in-line sheath and a delivery catheter associated with the in-line sheath. For example, the delivery catheter can be preloaded within the in-line sheath. The medical device delivery system can facilitate minimally invasive transcatheter delivery of a medical device to a target site, including a target site within the heart, including within a heart chamber, such as a left ventricle. The in-line sheath can have a sheath shaft comprising a proximal portion and an expandable distal portion, the expandable distal portion being configured to assume an expanded state and a collapsed state. While the expandable distal portion is in the expanded state, the delivery catheter can be preloaded within the in-line sheath such that at least a portion of a proximal portion of a catheter shaft of the delivery catheter can be received within the proximal portion of the sheath shaft and a distal portion of the catheter shaft can be received within the expandable distal portion. For example, the delivery catheter can be preloaded within a delivery catheter conduit of the in-line sheath.

In some instances, the medical device can be preloaded within the delivery catheter. The medical device can comprise a proximal narrow portion and a distal wide portion, the distal wide portion comprising a lateral cross-sectional size larger than that of the proximal narrow portion. The distal portion of the catheter shaft can have a larger lateral cross-sectional size than its proximal portion, for example to accommodate the distal wide portion of the medical device. Deploying the medical device can comprise translating the medical device distally relative to the delivery catheter and/or translating the delivery catheter distally relative to the in-line sheath, to position the distal wide portion of the medical device out distally of a distal end of the catheter shaft and to position the distal portion of the catheter shaft distally of a distal end of the sheath shaft, respectively. Retracting the medical device can comprise withdrawing the distal wide portion back into the distal portion of the catheter shaft. The distal portion of the catheter shaft can be subsequently retracted back into the expandable distal portion of the sheath shaft. The distal wide portion of the medical device can remain in the distal portion of the catheter shaft, and the distal portion of the catheter shaft can remain in the expandable distal portion of the in-line sheath, while the in-line sheath is retracted back through the anatomical pathways.

The delivery catheter comprising the medical device preloaded therethrough can be preloaded within the in-line sheath such that the distal portion of the catheter shaft and/or distal wide portion of the medical device are not advanced through the proximal portion of the sheath shaft for deployment and retraction of the medical device. Preloading the delivery catheter carrying the preloaded medical device in the in-line sheath can facilitate use of an in-line sheath comprising a sheath shaft comprising a proximal portion having a reduced profile and/or a proximal portion comprising a material different from that of the expandable distal portion. The reduced profile of the sheath shaft proximal portion can facilitate blood flow therearound. Use of different materials for the proximal portion and the expandable distal portion can facilitate providing desired torqueability of the proximal portion for navigation through tortuous anatomical pathways while allowing for desired accommodation of the distal wide portion of the medical device in the expandable distal portion. In some instances, the in-line sheath and the delivery catheter can be manufactured together such that the delivery catheter can be preloaded within the in-line sheath.

In some instances, the expandable distal portion of the sheath shaft can comprise a tubular configuration. In some instances, the expandable distal portion can comprise a pronged configuration. For example, the expandable distal portion can comprise a plurality of discrete elongate portions extending distally from the proximal portion of the sheath shaft. The plurality of discrete elongate portions can be disposed around a corresponding portion of the delivery catheter conduit, for example at least partially defining the delivery catheter conduit. Each discrete elongate portion can be configured to rotate outward about its proximal end to assume an expanded state so as to accommodate a distal wide portion of a medical device received within a distal portion of a catheter shaft.

The medical device can be configured to treat any number of conditions, including a variety of heart conditions. In some instances, the medical device can be configured to provide improved blood flow through the heart. For example, the medical device can comprise a mechanical circulatory support (MCS) mechanism. In some instances, the medical device can comprise a heart pump. In some instances, at least a portion of the medical device, such as the distal wide portion, can be positioned into a heart chamber, such as a left ventricle. The medical device can be configured to pump blood from the left ventricle into the aorta to thereby deliver oxygen-rich blood to the body. As described herein, the medical device delivery system can facilitate improved blood flow around the system during a deployment procedure. Improved blood flow can in turn facilitate performance of more complex procedures, such as procedures which require longer durations, including procedures for delivering a mechanical circulatory support (MCS) device.

The in-line sheath comprising the delivery catheter and medical device preloaded therein can be advanced through an outer expandable sheath positioned into the body. In some instances, the outer expandable sheath can be positioned into the body through an opening in an iliac artery or a femoral artery. The in-line sheath can be configured to provide mechanical column strength to reduce or prevent damage to the delivery catheter and/or medical device due to radial compression force exerted thereupon, such as by the outer expandable sheath as the delivery system is advanced through expandable portions of the outer expandable sheath. In some instances, the in-line sheath can be advanced to position the expandable distal portion of the sheath shaft distally of a distal end of the outer expandable sheath and into a desired chamber, vessel, and/or channel, such as an aorta, including a descending aorta or an abdominal aorta. After the expandable distal portion of the sheath shaft is advanced out of the outer expandable sheath, the delivery catheter can be advanced to position the distal portion of the catheter shaft distally of a distal end of the sheath shaft. The distal wide portion of the medical device can then be deployed from the catheter shaft and positioned at a target site. In some instances, the distal wide portion of the medical device can be positioned into the left ventricle.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

Although the target site is described primarily herein as within a heart chamber, such as a heart ventricle, it will be understood that the target site can be within any number of other organs, chambers, lumens and/or vessels. The medical device can be delivered to any number of other organs, chambers, lumens and/or vessels to treat conditions other than abnormalities of the heart.

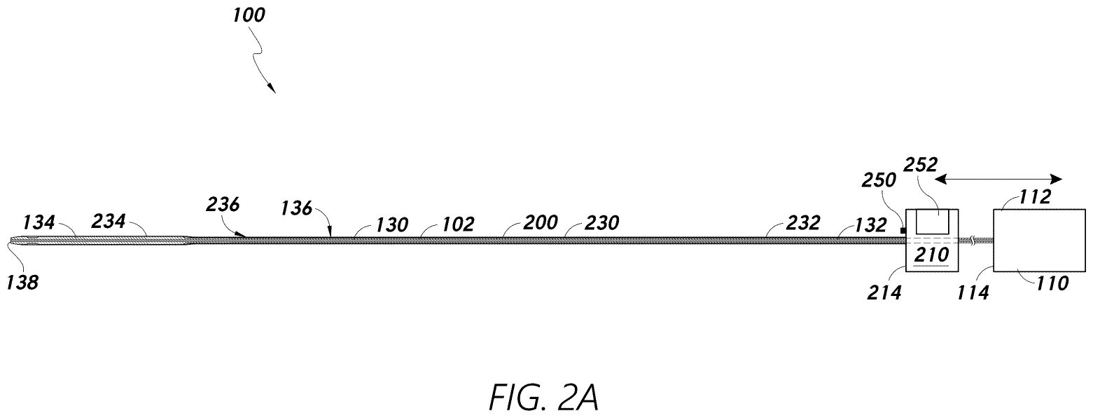

FIGS. 2A, 2B and 2C are side cross-sectional views of an example of a medical device delivery system 100. The medical device delivery system 100 can comprise a delivery catheter 102 and an in-line sheath 200 associated with the delivery catheter 102. The delivery catheter 102 can comprise a catheter hub 110 and a catheter shaft 130. The catheter shaft 130 can comprise a medical device conduit 136 extending through the catheter shaft 130, the medical device conduit 136 being configured to slidably receive at least a portion of a medical device. A proximal portion 132 of the catheter shaft 130 can be coupled to the catheter hub 110, such as a distal portion 112 of the catheter hub 110. For example, the catheter shaft 130 can extending distally from the catheter hub 110, including from a distal end 114 of the catheter hub 110. The in-line sheath 200 can comprise a sheath hub 210 and a sheath shaft 230. The sheath shaft 230 can comprise a delivery catheter conduit 236 extending through the sheath shaft 230 and configured to slidably receive at least a portion of the catheter shaft 130. The sheath shaft 230 can comprise a proximal portion 232 and an expandable distal portion 234. The sheath shaft 230, such as the proximal portion 232, can extend distally from the sheath hub 210. The proximal portion 232 of the sheath shaft 230 can be coupled to the sheath hub 210. As described in further detail herein, the expandable distal portion 234 can assume an expanded state configured to accommodate the distal portion 134 of the catheter shaft 130, and a collapsed state while the distal portion 134 of the catheter shaft 130 is advanced out of a distal end of and positioned distally of the expandable distal portion 234. In some instances, the medical device delivery system 100, including the delivery catheter 102 preloaded within the in-line sheath 200, can be advanced through an outer expandable sheath to facilitate delivery of the medical device to a target site.

The in-line sheath 200 can be associated with the delivery catheter 102. For example, the delivery catheter 102 can be preloaded within the in-line sheath 200 such that at least a portion of the proximal portion 132 of the catheter shaft 130 is received within the proximal portion 232 of the sheath shaft 230 and the distal portion 134 of the catheter shaft 130 is received within the expandable distal portion 234 of the sheath shaft 230 while the expandable distal portion 234 is in the expanded state. In some instances, the in-line sheath 200 and the delivery catheter 102 can be manufactured together such that the delivery catheter 102 is preloaded within the in-line sheath 200. The proximal portion 132 of the catheter shaft 130 can be preloaded within the proximal portion 232 of the sheath shaft 230 and the distal portion 134 of the catheter shaft 130 can be preloaded within the expandable distal portion 234 of the sheath shaft 230, such that the distal portion 134 of the catheter shaft 130 need not be slidably received within the proximal portion 232 of the sheath shaft 230. As described in further detail herein, the distal portion 134 of the catheter shaft 130 can have a larger lateral cross section than that of the proximal portion 132 of the catheter shaft 130. The proximal portion 232 of the sheath shaft 230 need not assume a larger lateral cross section to accommodate the larger distal portion 134 of the catheter shaft 130, as the distal portion 134 of the catheter shaft 130 need not be advanced through the proximal portion 232. The reduced profile of the proximal portion 232 of the sheath shaft 230 can facilitate blood flow therearound when positioned within the patient. As the proximal portion 232 does not accommodate the larger distal portion 134 of the catheter shaft 130, the proximal portion 232 can comprise at least a portion of which that is made of a material more rigid than that used for the expandable distal portion 234, thereby improving navigation through tortuous anatomical pathways.

Referring to FIG. 2A, the proximal portion 132 of the catheter shaft 130 can be configured to slidably receive at least a portion of a proximal narrow portion of the medical device. The distal portion 134 of the catheter shaft 130 can be configured to slidably receive a distal wide portion of the medical device and/or a portion of the proximal narrow portion of the medical device. The distal wide portion can comprise a lateral cross-sectional size larger than that of the proximal narrow portion. The lateral cross-section can be perpendicular or substantially perpendicular to a longitudinal axis of the medical device. For example, the distal wide portion can have a diameter wider than that of the proximal narrow portion of the medical device. In some instances, the medical device can be preloaded within the delivery catheter 102. For example, at least a portion of the proximal narrow portion of the medical device can be received within the proximal portion 132 of the catheter shaft 130 and the distal wide portion of the medical device can be received within the distal portion 134 of the catheter shaft 130. Deploying the medical device can comprise distally translating the medical device relative to the delivery catheter 102 such that the distal wide portion of the medical device is positioned distally out of an opening at a distal end 138 of the catheter shaft 130 and a portion of the proximal narrow portion of the medical device is received within the distal portion 134 of the catheter shaft 130.

In some instances, the distal portion 134 of the catheter shaft 130 can have a lateral cross section sized to accommodate the distal wide portion of the medical device. For example, the distal portion 134 can comprise a lateral cross section larger than that of the proximal portion 132. The lateral cross-section can be perpendicular or substantially perpendicular to a longitudinal axis of the catheter shaft 130. The distal portion 134 can have a diameter wider than that of the proximal portion 132. For example, an outer diameter and/or an inner diameter, including a diameter of the medical device conduit 136, of the proximal portion 132 can be smaller than that of the distal portion 134.

In some instances, while in the expanded state, the expandable distal portion 234 of the sheath shaft 230 can have a lateral cross section sized to accommodate the distal portion 134 of the catheter shaft 130. For example, while in the expanded state, the expandable distal portion 234 can comprise a lateral cross section larger than that of the proximal portion 232. The lateral cross-section can be perpendicular or substantially perpendicular to a longitudinal axis of the sheath shaft 230. The expandable distal portion 234 can have a diameter wider than that of the proximal portion 232. For example, an outer diameter and/or an inner diameter, including a diameter of the delivery catheter conduit 236, of the proximal portion 232 can be smaller than that of the expandable distal portion 234.

In some instances, the sheath hub 210 can comprise an outer sheath engagement feature 250 configured to engage with an outer expandable sheath to prevent or reduce distal and proximal translation of the in-line sheath 200 relative to the outer expandable sheath. In some instances, the outer sheath engagement feature 250 can be configured to prevent or reduce rotational movement of the in-line sheath 200 relative to the outer expandable sheath, such as rotation around a longitudinal axis of the in-line sheath 200. In some instances, the outer sheath engagement feature 250 can be configured to engage with an outer sheath hub of the outer expandable sheath. For example, the outer sheath engagement feature 250 can comprise at least a portion on a distal end 214 of the sheath hub 210. The distal end 214 of the sheath hub 210 can be positioned against or proximate to a proximal end of the outer sheath hub such that the outer sheath engagement feature 250 can be triggered, activated, and/or engaged to fix the relative rotational and translational positions of the outer expandable sheath and the in-line sheath 200.

In some instances, the sheath hub 210 can comprise a catheter engagement feature 252 configured to engage with a portion of the delivery catheter 102 to prevent or reduce distal and proximal translation of the delivery catheter 102 relative to the in-line sheath 200. In some instances, the catheter engagement feature 252 can be configured to prevent or reduce rotational movement of the delivery catheter 102, such as rotation around a longitudinal axis of the delivery catheter 102. In some instances, the catheter engagement feature 252 can be configured to frictionally contact a portion of the catheter shaft 130 extending through the sheath hub 210. The catheter engagement feature 252 can be triggered, activated, and/or engaged such that one or more portions thereof can frictionally contact a portion of the catheter shaft 130. For example, the catheter engagement feature 252 can be engaged with a portion of the catheter shaft 130 extending through the sheath hub 210 after the distal portion 134 of the catheter shaft 130 is at a desired position distal of the expandable distal portion 234.

FIGS. 2B and 2C show the expandable distal portion 234 of the sheath shaft 230 in further detail. As described herein, the expandable distal portion 234 can be configured to assume an expanded state or a collapsed state. While in the expanded state, the expandable distal portion 234 can be configured to slidably receive the distal portion 134 of the catheter shaft 130. For example, while in the expanded state, the expandable distal portion 234 can comprise an outer diameter and/or an inner diameter that is larger than that of the proximal portion 232 of the sheath shaft 230. FIGS. 2B and 2C show the expandable distal portion 234 in the expanded state, while the distal portion 134 of the catheter shaft 130 is received through the expandable distal portion 234. While the expandable distal portion 234 of the sheath shaft 230 is in the collapsed state, a portion of the proximal portion 132 of the catheter shaft 130 can be received through the expandable distal portion 234. For example, the expandable distal portion 234 of the sheath shaft 230 can assume the collapsed state after the distal portion 134 of the catheter shaft 130 is advanced distally out of an opening at a distal end 238 of the sheath shaft 230 and a portion of the proximal portion 132 of the catheter shaft 130 is received in the expandable distal portion 234.

In some instances, the expandable distal portion 234 of the sheath shaft 230 can comprise a tubular configuration. In some instances, the expandable distal portion 234, while in the expanded state, can comprise a cylindrical shape.

Referring to FIG. 2C, in some instances, a proximally oriented portion 242 of the expandable distal portion 234 can comprise a sloped profile to provide a transition from the narrower proximal portion 232 to a wider lateral cross section of the expandable distal portion 234. In some instances, the sloped profile can provide a space between the catheter shaft 130 received within the delivery catheter conduit 236 and the sheath shaft 230. In some instances, the space can be configured to accommodate a portion of a guidewire configured to be threaded through the medical device to facilitate deployment of the medical device. In some instances, a distally oriented portion 244 of the expandable distal portion 234 can provide a space between the catheter shaft 130 received within the delivery catheter conduit 236 and the sheath shaft 230, such as the distal portion 134 of the catheter shaft 130. The space can be configured to accommodate a portion of a guidewire configured to be threaded through the medical device to facilitate deployment of the medical device.

In some instances, the medical device can be pre-loaded within the delivery catheter 102 such that the distal wide portion of the medical device is disposed within the distal portion of the 134 of the catheter shaft 130 and the proximal narrow portion of the medical device is disposed within the proximal portion 132 of the catheter shaft 130. It is noted that the medical device is not shown for simplicity. The delivery catheter 102 comprising the medical device preloaded therein can be preloaded within the in-line sheath 200 such that the proximal portion 132 of the catheter shaft 130 is disposed within the proximal portion 232 of the sheath shaft 230 and the distal portion 134 of the catheter shaft 130 is received within the expandable distal portion 234. The delivery catheter 102 comprising the medical device preloaded therein can be translated distally relative to the in-line sheath 200 such that the distal portion 134 of the catheter shaft 130 carrying the preloaded medical device can be extended out of the in-line sheath 200. For example, deploying the medical device can comprise advancing the delivery catheter 102 distally relative to the in-line sheath 200 to position the distal portion 134 of the catheter shaft 130 distally of the distal end 238 of the sheath shaft 230. Subsequently, the medical device can be advanced distally relative to the delivery catheter 102 to position the distal wide portion of the medical device distally of a distal end of the catheter shaft 130.

In some instances, the proximal portion 232 of the sheath shaft 230 can comprise a material different from that of the expandable distal portion 234. In some instances, the proximal portion 232 can comprise a flexible material. In some instances, the expandable distal portion 234 can comprise a material that is both flexible and expandable. In some instances, the sheath shaft 230 can comprise a laser cut hypotube that provides desired flexibility, torqueability, and/or expandability. In some instances, the hypotube can comprise stainless steel and/or a shape memory material (e.g., nitinol). In some instances, the sheath shaft 230 can comprise a polyimide (e.g., nylon), and/or a thermoplastic elastomer (e.g., polyether block amide). In some instances, one or more portions of the sheath shaft 230 can comprise a radiopaque material to facilitate visualization of the sheath shaft 230 during deployment, including at a distal end 238 of the sheath shaft 230. In some instances, one or more portions of the sheath shaft 230 can comprise a hydrophilic coating thereon to facilitate advancement of the sheath shaft 230, including through an outer expandable sheath.

In alternative instances, a sheath shaft can comprise a distal portion that is not expandable. The distal portion of the sheath shaft may not expand and/or collapse. For example, the non-expandable distal portion can have a larger lateral cross section, such as compared to a proximal portion of the sheath shaft, configured to accommodate a distal portion of a catheter shaft carrying a distal wide portion of a medical device therein. The non-expandable distal portion of the sheath shaft may not collapse after the distal portion of the catheter shaft is advanced distally out of the distal portion of the sheath shaft. For example, the non-expandable distal portion can maintain or substantially maintain its configuration, such as the larger cross-sectional size as compared to the proximal portion of the sheath shaft, with or without the distal portion of the catheter shaft received through the non-expandable distal portion.

In some instances, the non-expandable distal portion can have the same configuration as that of the expandable distal portion 234 of the sheath shaft 230 in the expanded state as described with reference to FIG. 2. The non-expandable distal portion can comprise a size, including a cross-sectional size, and shape as that of the expandable distal portion 234 in the expanded state. For example, the non-expandable distal portion can comprise a tubular configuration, including a cylindrical shape. In some instances, the non-expandable distal portion can maintain or substantially maintain the cylindrical shape with or without the distal portion of the catheter shaft received through the non-expandable distal portion. In some instances, a proximally oriented portion of the non-expandable distal portion can comprise a sloped profile to provide a transition from the narrower proximal portion to a wider lateral cross section of the non-expandable distal portion.

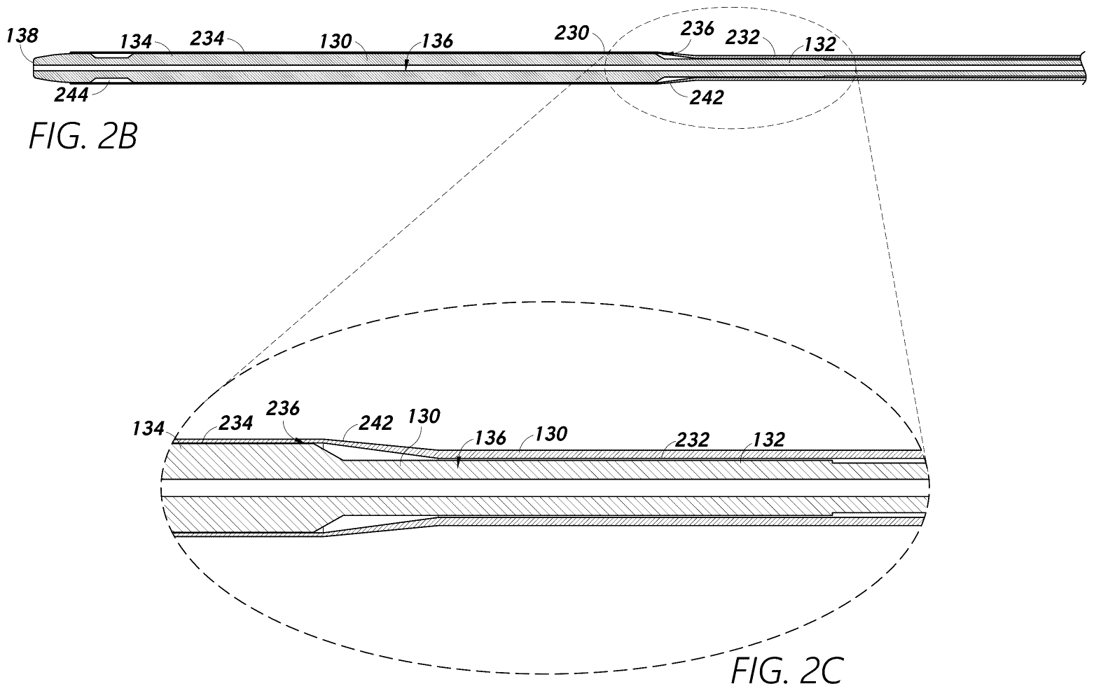

FIGS. 3A, 3B and 3C are side cross-sectional views of a sheath delivery system 300 comprising the medical device delivery system 100 described with reference to FIG. 2 positioned through an outer expandable sheath 302. Referring to FIG. 3A, the outer expandable sheath 302 can comprise an outer sheath hub 310 and an outer sheath shaft 330. The outer sheath shaft 330 can comprise a proximal portion 332 and a distal portion 334. The outer sheath shaft 330 can be coupled to the sheath hub 310, for example extending distally from the outer sheath hub 310, including from a distal end 314 of the sheath hub 310. The medical device delivery system 100 can comprise the delivery catheter 102 preloaded within the in-line sheath 200. At least a portion of the sheath shaft 230 comprising at least a portion of the catheter shaft 130 preloaded therethrough can be advanced through the outer expandable sheath 302. For example, the sheath shaft 230 comprising at least a portion of the catheter shaft 130 preloaded through the sheath shaft 230 can be advanced through the outer sheath hub 310 and the outer sheath shaft 330. In some instances, the medical device can be pre-loaded within the delivery catheter 102 such that the distal wide portion of the medical device is disposed within the distal portion 134 of the of the catheter shaft 130 and at least a portion of the proximal narrow portion of the medical device is disposed within the proximal portion 132 of the catheter shaft 130. The delivery catheter 102 comprising the medical device preloaded therein can be preloaded within the in-line sheath 200.

The sheath shaft 230 can provide structural support for and/or protect the catheter shaft 130, and the medical device carried by the catheter shaft 130, while the sheath shaft 230 is advanced through the outer sheath hub 310 and outer sheath shaft 330. The outer sheath hub 310 can comprise one or more seals, including one or more hemostatic seals. For example, the sheath shaft 230 can be advanced through a first seal 320 and a second seal 322 housed within the outer sheath hub 310, as the in-line sheath 200 is translated distally relative to the outer expandable sheath 302. The sheath shaft 230 can provide mechanical column strength and/or protection for the catheter shaft 130 and medical device preloaded therein as the sheath shaft 230 is advanced through the seals 320, 322.

In some instances, the outer sheath shaft 330 can comprise at least a portion that is expandable. For example, at least a portion of the outer sheath shaft 330 can be in a collapsed state prior to insertion of the delivery system 100 through the outer sheath shaft 330. Corresponding expandable portions of the outer sheath shaft 330 can assume an expanded state as the sheath shaft 230 is advanced therethrough. Contact between the corresponding expandable portions of the outer sheath shaft 330 and the sheath shaft 230 can expand the outer sheath shaft 330 such that the outer sheath shaft 330 can assume the expanded state. The sheath shaft 230 can provide mechanical column strength for the catheter shaft 130 and medical device as the sheath shaft 230 is advanced through the outer sheath shaft 330.

FIG. 3A shows the expandable distal portion 234 of the sheath shaft 230 disposed distally of a distal end 338 of the outer sheath shaft 330. For example, the in-line sheath 200 can be translated distally relative to the outer expandable sheath 302 such that the expandable distal portion 234 is advanced through the outer sheath shaft 330 from a proximal end 336 to the distal end 338. The expandable distal portion 234 can be advanced distally out of an opening 340 at the distal end 338. For example, a portion of the proximal portion 232 of the sheath shaft 230 can be positioned through the opening 340 and another portion of the proximal portion 232 of the sheath shaft 230 can be disposed within the outer sheath shaft 330. Expanded portions of the outer sheath shaft 330 can assume a reduced profile as the expandable distal portion 234 of the sheath shaft 230 is advanced distally out of the outer sheath shaft 330. In some instances, a reduction in the profile of the outer sheath shaft 330 can facilitate blood flow therearound, thereby providing improved blood flow to tissues during the procedure, and reducing complications and/or facilitating procedures with increased duration. As described herein, at least a portion of the outer sheath shaft 330 can be in a collapsed state prior to the insertion of the medical device delivery system 100 through the outer sheath shaft 330. The expandable portion of the outer sheath shaft 330 can expand to accommodate the sheath shaft 230 as the sheath shaft 230 is advanced through the outer sheath shaft 330. The collapsed portion can exert friction and radial compressive force upon the sheath shaft 230 during advancement of the sheath shaft 230. The sheath shaft 230 can be configured to provide desired mechanical column strength to prevent or reduce damage to the catheter shaft 130 and/or medical device received therein.

The outer sheath engagement feature 250 comprising at least a portion on a distal end 214 of the sheath hub 210 can be configured to engage with the outer expandable sheath 302 to prevent or reduce distal and proximal translation and/or rotation of the in-line sheath 200 relative to the outer expandable sheath 302. The outer sheath engagement feature 250 can be engaged with an in-line sheath engagement feature 350 of the outer sheath hub 310. The in-line sheath engagement feature 350 can comprise at least a portion on the proximal end 312 of the outer sheath hub 310. The outer sheath engagement feature 250 can engage with the in-line sheath engagement feature 350 so as to secure the relative translational and/or rotational positions of the outer expandable sheath 302 and the in-line sheath 200 after the expandable distal portion 234 is at a desired position distal of the distal end 338 of the outer sheath shaft 330. In some instances, the distal end 214 of the sheath hub 210 can be positioned against or proximate to the proximal end 312 of the outer sheath hub 310 such that the outer sheath engagement feature 250 can be engaged with the in-line sheath engagement feature 350 of the outer sheath hub 310.

The catheter engagement feature 252 can be configured to frictionally engage with a portion of the delivery catheter 102 to prevent or reduce distal and proximal translation, and/or rotation, of the delivery catheter 102 relative to the in-line sheath 200. The catheter engagement feature 252 can be triggered, activated, and/or engaged such that one or more portions thereof can frictionally contact a portion of the catheter shaft 130 extending through the sheath hub 210 after the distal portion 134 of the catheter shaft 130 is at a desired position distal of the expandable distal portion 234. In some instances, the delivery catheter 102 can be advanced relative to the in-line sheath 200 until the distal end 114 of the catheter hub 110 is in contact with the proximal end 212 of the sheath hub 210.

The arrows in FIG. 3A show distal and proximal translation of the in-line sheath 200 and the delivery catheter 102. For example, the top arrow shows distal and proximal translation of the in-line sheath 200 relative to the outer expandable sheath 302. The bottom arrow shows distal and proximal translation of the delivery catheter 102 relative to the in-line sheath 200. Referring to FIG. 3B, the distal portion 134 of the catheter shaft 130 is shown as being disposed distally of a distal end 238 of the sheath shaft 230. FIG. 3C shows in further detail the features shown within the dotted oval in FIG. 3B. In some instances, deploying the medical device can comprise translating the delivery catheter 102 distally relative to the in-line sheath 200 to advance the distal portion 134 of the catheter shaft 130 through an opening 240 at the distal end 238 of the sheath shaft 230. The distal wide portion of the medical device can subsequently be advanced through a distal end 138 of the catheter shaft 130.

As described herein, after the expandable distal portion 234 is at a desired position distal of the distal end 338 of the outer sheath shaft 330, the outer sheath engagement feature 250 can be engaged with the in-line sheath engagement feature 350. The distal end 214 of the sheath hub 210 can be positioned against or proximate to the proximal end 312 of the outer sheath hub 310 such that the in-line sheath engagement feature 350 can be engaged with the outer sheath engagement feature 250 of the sheath hub 210. After the distal portion 134 of the catheter shaft 130 is at a desired position distal of the distal end 238 of the sheath shaft 230, the catheter engagement feature 252 can be frictionally contact a portion of the catheter shaft 130 extending through the sheath hub 210.

FIG. 3C shows in further detail the distal portion 134 of the catheter shaft 130 disposed distally of the distal end 238 of the sheath shaft 230 and the expandable distal portion 234 of the sheath shaft 230 disposed distally of the distal end 338 of the outer sheath shaft 330. A portion of the proximal portion 232 of the sheath shaft 230 can be positioned through the opening 340 at the distal end 338 of the outer sheath shaft 330 and another portion of the proximal portion 232 of the sheath shaft 230 can be disposed within the outer sheath shaft 330. A portion of the proximal portion 132 of the catheter shaft 130 can be positioned through the opening 240 at the distal end 238 of the sheath shaft 230 and another portion of the proximal portion 132 of the catheter shaft 130 can be disposed within the expandable distal portion 234 of the sheath shaft 230. Corresponding portions of the expandable distal portion 234 can assume the collapsed state as the distal portion 134 of the catheter shaft 130 is advanced distally out of the expandable distal portion 234 and a portion of the proximal portion 132 of the catheter shaft 130 is received through the expandable distal portion 234. In some instances, the expandable distal portion 234 assuming the collapsed state can facilitate blood flow therearound to thereby provide improved blood flow to tissues during the procedure.

The medical device can be translated distally relative to the delivery catheter 102 and the in-line sheath 200 to advance the distal wide portion of the medical device out of an opening at the distal end 138 of the catheter shaft 130. For example, the distal wide portion of the medical device can be positioned distally of the distal portion 134 of the catheter shaft 130 at a target site, such as within a heart chamber, including a heart ventricle.

The medical device can be retracted for withdrawal from the target site. For example, the medical device can be translated proximally relative to the delivery catheter 102 to position the distal wide portion of the medical device back into the distal portion 134 of the catheter shaft 130. The delivery catheter 102 can then be translated proximally relative to the in-line sheath 200 to position the distal portion 134 of the catheter shaft 130 back into the expandable distal portion 234 of the sheath shaft 230. The in-line sheath 200, delivery catheter 102 and medical device can then be withdrawn together. The distal wide portion of the medical device and the distal portion 134 of the catheter shaft 130 can remain in the expandable distal portion 234 while the in-line sheath 200, delivery catheter 102 and medical device are retracted back through the outer expandable sheath 302. The distal portion 134 of the catheter shaft 130 and/or distal wide portion of the medical device may not need to be advanced through the proximal portion 232 of the sheath shaft 230 for deployment and retraction of the medical device. The proximal portion 232 of the sheath shaft 230 can thereby have a reduced profile and/or comprise a material different from that of the expandable distal portion 234.

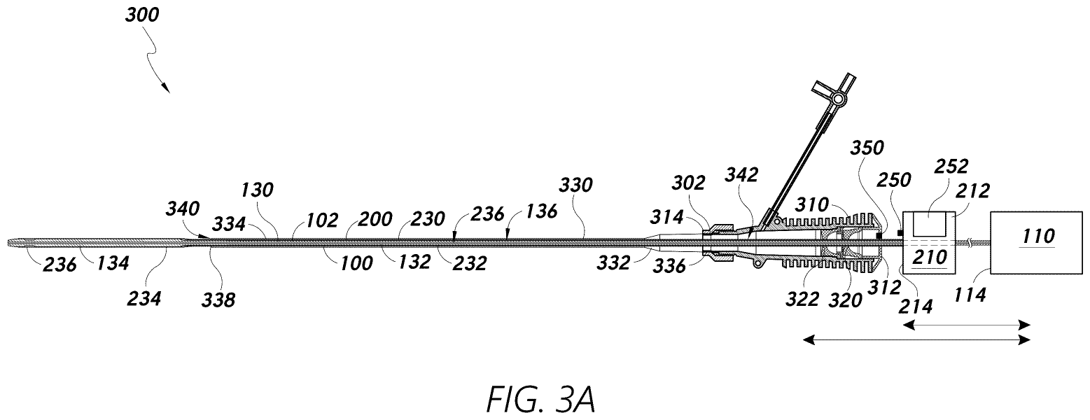

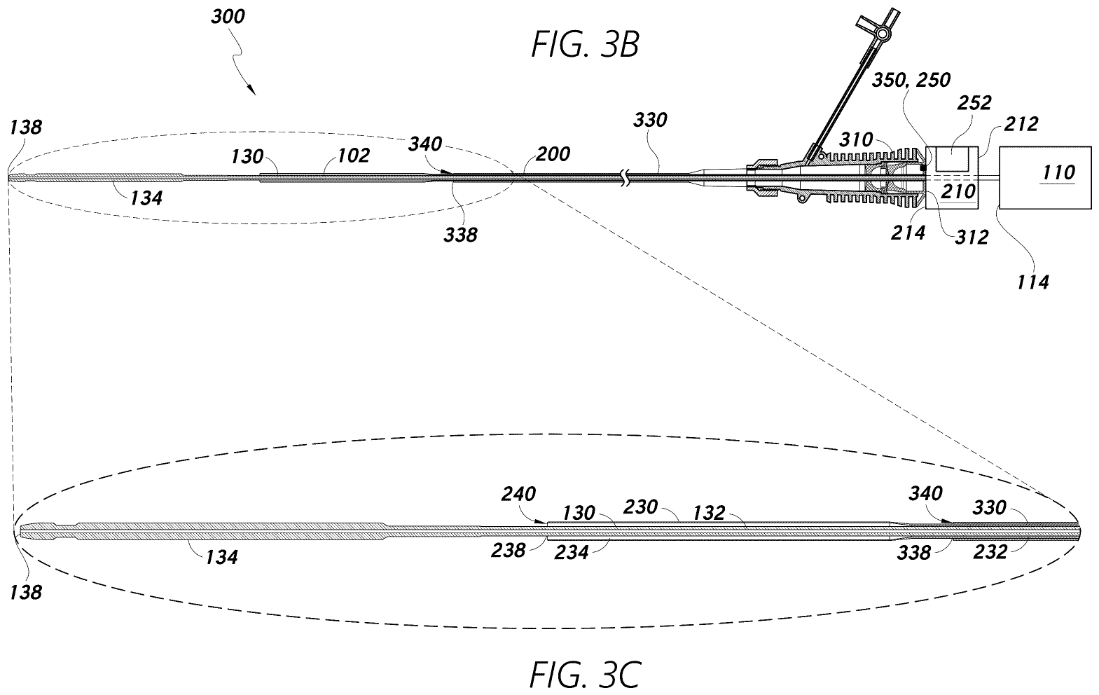

FIGS. 4 and 5 are perspective views of examples of sheath shafts comprising expandable distal portions made of different materials, where the expandable distal portions are shown in expanded states. FIG. 4 shows an example of a sheath shaft 400 that includes an expandable distal portion 404 comprising an expandable coil 408. The expandable coil 408 can facilitate expansion and/or contraction of the expandable distal portion 404. The sheath shaft 400 can comprise a delivery catheter conduit 406 extending through the sheath shaft 400 and configured to receive at least a portion of a delivery catheter. The delivery catheter can be preloaded within the delivery catheter conduit 406 such that a distal portion of a catheter shaft of the delivery catheter can be disposed within the expandable distal portion 404 while the expandable distal portion 404 is in an expanded state, for example within the portion of the delivery catheter conduit 406 extending through the expandable distal portion 404. The expandable distal portion 404 can comprise a proximally oriented portion 410 and a distally oriented portion 412. In some instances, the proximally oriented portion 410 can comprise a transition from a proximal portion 402 of the sheath shaft 400 to the expandable distal portion 404. For example, the transition can comprise a lateral cross section that increases along a direction extending from proximally oriented portion 410 to the distally oriented portion 412. The lateral cross section can be perpendicular or substantially perpendicular to a longitudinal axis of the sheath shaft 400.

The expandable distal portion 404 can comprise the expandable coil 408 along a longitudinal portion. The expandable coil 408 can extend along at least a portion of a longitudinal dimension, including along an entire longitudinal dimension, such as an entire length of the expandable distal portion 404. The longitudinal dimension can extend along the longitudinal axis of sheath shaft 400. In some instances, coils of the expandable coil 408 can be around a corresponding portion of the delivery catheter conduit 406. For example, the expandable distal portion 404 can comprise an expandable coil 408 having its coils positioned around the portion of the delivery catheter conduit 406 of the expandable distal portion 404. In some instances, coils of the expandable coil 408 positioned around the delivery catheter conduit 406 can define at least in part the delivery catheter conduit 406. In some instances, the expandable distal portion 404 comprising the expandable coil 408 can comprise a tubular configuration, including a cylindrical shape. For example, coils of the expandable coil 408 can form a cylindrical shape of the expandable distal portion 404.

FIG. 5 shows an example of a sheath shaft 500 that includes an expandable distal portion 504 comprising a shape memory material 508 configured to facilitate expansion and/or contraction of the expandable distal portion 504. The expandable distal portion 504 is shown in an expanded state, such as while a distal portion of a catheter shaft of a delivery catheter is preloaded therein. The sheath shaft 500 can comprise a delivery catheter conduit 506 extending through the sheath shaft 500. For example, while the catheter shaft is preloaded within the delivery catheter conduit 506, the distal portion of the catheter shaft can be disposed within the portion of the delivery catheter conduit 506 extending through the expandable distal portion 504. At least a portion of the expandable distal portion 504 can comprise the shape memory material 508. The shape memory material 508 can be along at least a portion of a longitudinal dimension, including along an entire longitudinal dimension, such as an entire length of the expandable distal portion 504. The longitudinal dimension can extend along a longitudinal axis of the sheath shaft 500. Any number of shape memory materials can be used, including for example, nitinol. In some instances, the expandable distal portion 504 can comprise a tubular configuration, including a cylindrical shape. For example, the expandable portion 504 comprising the shape memory material 508 can form a cylindrical shape.

The expandable distal portion 504 can comprise a proximally oriented portion 510 and a distally oriented portion 512. In some instances, the proximally oriented portion 510 can comprise a transition from a proximal portion 502 of the sheath shaft 500 to the expandable distal portion 504. For example, the transition can comprise a lateral cross section that increases along a direction extending from proximally oriented portion 510 to the distally oriented portion 512. The lateral cross section can be perpendicular or substantially perpendicular to the longitudinal axis of the sheath shaft 500.

The sheath shafts 400, 500 described with reference to FIGS. 4 and 5 can comprise a proximal portion 402, 502 which may or may not comprise the same material as the respective expandable distal portion 404, 504. In some instances, the proximal portion 402 of the sheath shaft 400 described with reference to FIG. 4 can comprise a material different from the expandable distal portion 404. In some instances, the proximal portion 502 of the sheath shaft 500 described with reference to FIG. 5 can comprise the same material as the expandable distal portion 504.

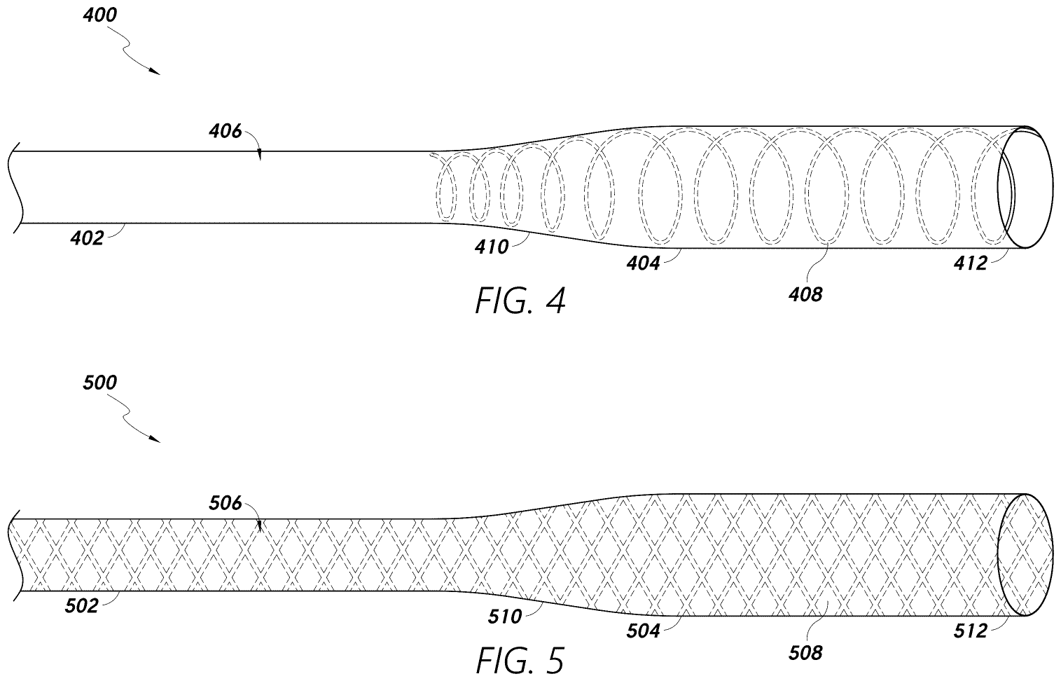

FIG. 6 is a perspective view of an example of a sheath shaft 600 that includes an expandable distal portion 604 comprising a plurality of discrete elongate portions 608 circumferentially disposed around a delivery catheter conduit 606. The plurality of discrete elongate portions 608 can partially define a corresponding portion of the delivery catheter conduit 606, such as a portion of the delivery catheter conduit 606 extending through the expandable distal portion 604. The delivery catheter conduit 606 can be configured to slidably receive at least a portion of a catheter shaft of a delivery catheter. The plurality of discrete elongate portions 608 can be coupled to a proximal portion 602 the sheath shaft 600. Each discrete elongate portion 608 can comprise a proximally oriented end portion 610 and a distally oriented end portion 612. The proximally oriented portions 610 can be coupled to one another. For example, the proximally oriented portions 610 can be coupled to the proximal portion 602 of the sheath shaft 600. In some instances, each of the plurality of discrete elongate portions 608 can extend distally from the proximal portion 602, for example a respective proximal end 614 of each discrete elongate portion 608 can be adjacent to a distal end of the proximal portion 602.

Each of the plurality of discrete elongate portions 608 can be spaced from adjacent discrete elongate portions 608 along at least a portion of a respective longitudinal dimension, thereby facilitating movement of the portion of the discrete elongate portions 608 away from adjacent discrete elongate portions 608. The distally oriented end portions 612 can be spaced from one another, such that while the expandable distal portion 604 is in the expanded state, the distally oriented end portions 612 can be configured to be disposed further apart from one another than while the expandable distal portion 604 is in the collapsed state. In some instances, each of the plurality of discrete elongate portions 608 can be spaced from the adjacent discrete elongate portions 608 along an entire length of the respective discrete elongate portion 608. For example, each of the plurality of discrete elongate portions 608 can rotate about its proximal end 614 to move away from, and back toward, adjacent discrete elongate portions 608, such that the expandable distal portion 604 can assume the expanded state and collapsed state, respectively. Each of the plurality of discrete elongate portions 608 can rotate about its proximal end 614 to accommodate a distal portion of a catheter shaft positioned through the expandable distal portion 604, such that a distally oriented end portion 612 of a discrete elongate portion 608 can be further away from adjacent distally oriented end portions 612. Each of the plurality of discrete elongate portions 608 can rotate back toward one another while the distal portion of the catheter shaft is positioned distally of the expandable distal portion 604, for example positioning the distally oriented end portions 612 closer to one another. In some instances, the expandable distal portion 604 can assume the collapsed state while the distal portion of the catheter shaft is positioned distally of the expandable distal portion 604. FIG. 6 shows the expandable distal portion 604 in the collapsed state.

Referring again to FIG. 6, each discrete elongate portion 608 can comprise a first portion 618 and a second portion 620, the second portion 620 being distal of the first portion 618. Each of the first portions 618 can have an angled orientation relative to the longitudinal axis of the sheath shaft 600. The first portion 618 can extend from the proximal portion 602 of the sheath shaft 600 at an angle relative to a longitudinal axis of the sheath shaft 600 to provide a transition from a smaller lateral cross section of the proximal portion 602 to a larger lateral cross section of the expandable distal portion 604. For example, the first portion 618 can comprise a sloped profile to provide a transition from a smaller lateral cross section of the proximal portion 602 to a larger lateral cross section of the expandable distal portion 604. In some instances, the second portion 620 can extend from the first portion 618 to a distal end 616 of the respective discrete elongate portion 608 along an axis parallel or substantially parallel to the longitudinal axis of the sheath shaft 600. A lateral cross section of the sheath shaft 600 at the proximal ends 614 of the discrete elongate portions 608 can be smaller than that at the distal ends 616 of the discrete elongate portions 608. For example, an inner diameter and/or outer diameter of the sheath shaft 600 at the proximal ends 614 can be shorter than that at the distal ends 616.

Although FIG. 6 shows four discrete elongate portions 608 circumferentially disposed around the delivery catheter conduit 606, it will be understood that an expandable distal portion can comprise fewer or more elongate portions. The discrete elongate portions can have any number of configurations. In some instances, a discrete elongate portion can have a rod configuration. In some instances, a discrete elongate portion can comprise one or more curved surface portions at least partially defining a delivery catheter conduit. For example, a discrete elongate portion can comprise a first surface oriented toward the delivery catheter conduit and extending along at least a longitudinal dimension of the expandable distal portion. The first surface can comprise a curvature along a lateral dimension, such as along a dimension perpendicular or substantially perpendicular to the longitudinal dimension, and configured to at least partially define the delivery catheter conduit. In some instances, the curvature can comprise a segment of a circle. In some instances, each discrete elongate portion can be identical. The discrete elongate portions can be evenly spaced around a delivery catheter conduit. For example, the expandable distal portion 604 can comprise four identical discrete elongate portions 608 extending distally from the proximal portion 602 and can be evenly spaced around the delivery catheter conduit 606.

The proximal portion 602 and the expandable distal portion 604 may or may not comprise different materials. FIG. 6 shows the proximal portion 602 of the sheath shaft 600 comprising a material different from that of the expandable distal portion 604. For example, the proximal portion 602 comprising a first material can be attached, bonded and/or fitted to the expandable distal portion 604. Alternatively, the sheath shaft 600 can be an integral component. In some instances, the sheath shaft 600 can be an integral unit comprising a uniform material throughout.

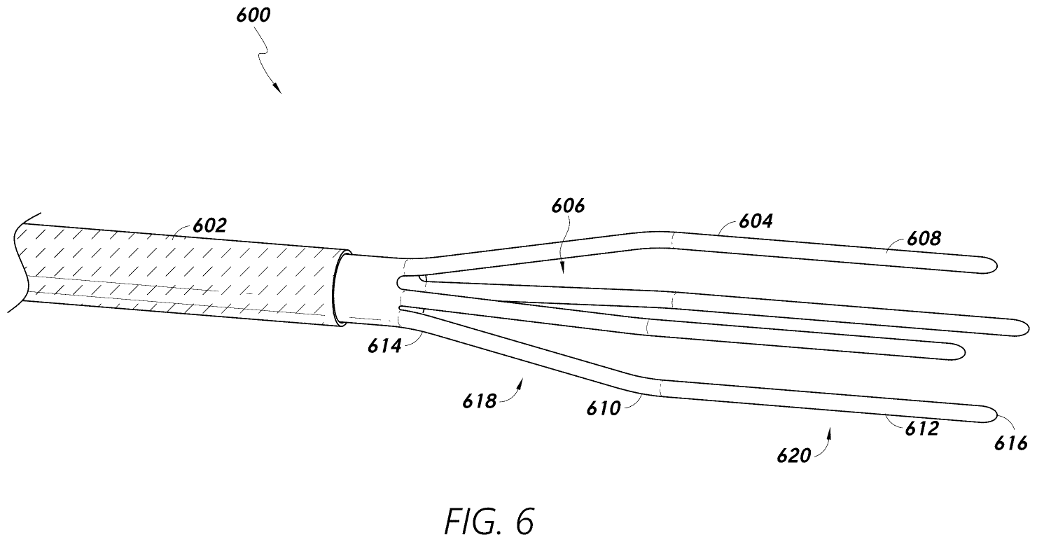

FIG. 7A is a side view, and FIG. 7B is a side cross-sectional view, of an example of a sheath shaft 700 with an expandable distal portion 704 comprising a plurality of discrete elongate portions 708, in a collapsed state and in an expanded state, respectively. Referring to FIG. 7A, while in the collapsed state, each of the plurality of discrete elongate portions 708 can extend distally along an axis parallel or substantially parallel to a longitudinal axis of the sheath shaft 700. The plurality of discrete elongate portions 708 can be circumferentially disposed around a delivery catheter conduit 706 of the sheath shaft 700. FIG. 7A does not show a catheter shaft disposed through the delivery catheter conduit 706. The plurality of discrete elongate portions 708 can be coupled to a proximal portion 702 of the sheath shaft 700. Each discrete elongate portion 708 can comprise a proximally oriented end portion 710 and a distally oriented end portion 712. The proximally oriented portions 710 can be coupled to the proximal portion 702 of the sheath shaft 700. For example, each of the plurality of discrete elongate portions 708 can extend distally from the proximal portion 702 along the axis parallel or substantially parallel to the longitudinal axis of the sheath shaft 700.

While the expandable distal portion 704 is in the collapsed state, the expandable distal portion 704 can have a uniform or substantially uniform lateral cross-sectional area along a longitudinal dimension. The lateral cross section can be perpendicular or substantially perpendicular to the longitudinal axis of the sheath shaft 700. The longitudinal dimension can be parallel or substantially parallel to the longitudinal axis of the sheath shaft 700. While in the collapsed state, the expandable distal portion 704 can have the same or similar cross-sectional size at proximal ends 714 as at distal ends 716 of each of the plurality of discrete elongate portions 708. In some instances, the expandable distal portion 704 can have a uniform inner and/or outer diameter along a longitudinal dimension, including an entire length of the expandable distal portion 704. For example, while in the collapsed state, the expandable distal portion 704 can have a uniform or substantially uniform inner and/or outer diameter along an entire or substantially entire length. The inner and/or outer diameter at a proximal end 718 of the expandable distal portion 704 is the same as that at a distal end 720 of the expandable distal portion 704.

Referring to FIG. 7B, the expandable distal portion 704 is shown in an expanded state while a distal portion 754 of a catheter shaft 750 is disposed through the expandable distal portion 704. For example, the catheter shaft 750 is disposed through the delivery catheter conduit 706 of the sheath shaft 700. A proximal portion 752 of the catheter shaft 750 is shown as being disposed within the proximal portion 702 of the sheath shaft 700. Each of the plurality of discrete elongate portions 708 can be rotated outward about a respective proximal end 714 in the expanded state to accommodate the distal portion 754 of the catheter shaft 750. The distal ends 716 of the plurality of elongate portions 708 can be spaced further apart from one another while the expandable distal portion 704 is in the expanded state than in the collapsed state. For example, the distal portion 754 of the catheter shaft 750 can push against the proximally oriented portions 710 of each of the plurality of discrete elongate portions 708, thereby causing the plurality of discrete elongate portions 708 to rotate about its proximal end 714 away from one another. A proximal end 756 of the catheter shaft distal portion 754 can be proximate or adjacent to proximal ends 714 of the discrete elongate portions 708 and a distal end 758 of the catheter shaft distal portion 754 can be oriented toward the distal ends 716 of the discrete elongate portions 708.

In some instances, the catheter shaft 750 can be advanced relative to the sheath shaft 700 to deploy the catheter shaft 750. The expandable distal portion 704 can assume the collapsed state when the catheter shaft distal portion 754 is positioned distally of the distal end 720 of the expandable distal portion 704.

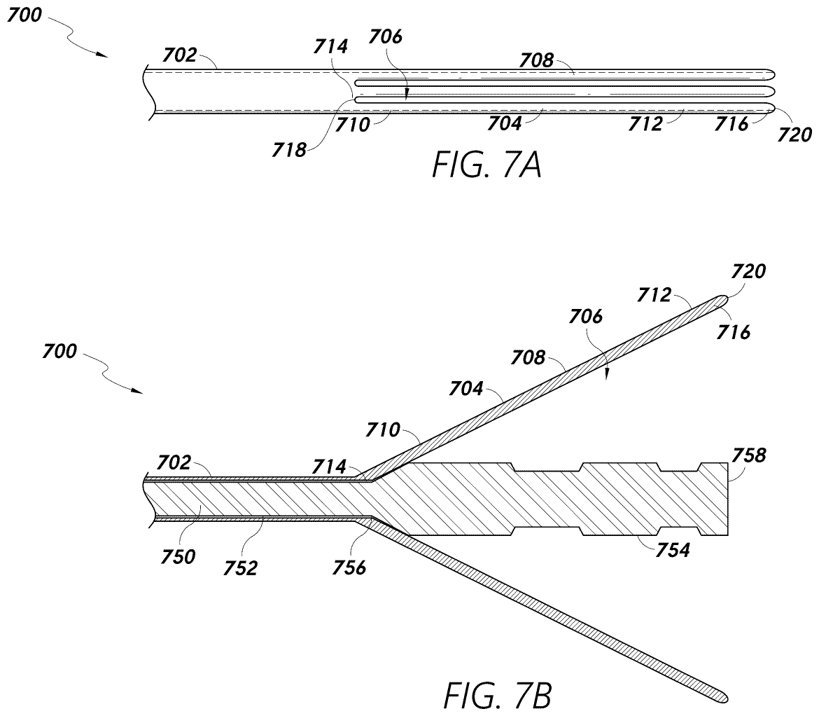

FIG. 8A is a side view, and FIG. 8B is a side cross-sectional view, of an example of a sheath shaft 800 comprising a plurality of discrete elongate portions 808 in a collapsed state and in an expanded state, respectively. Referring to FIG. 8A, the sheath shaft 800 is shown in the collapsed state. Each discrete elongate portion 808 can comprise a proximally oriented end portion 810 and a distally oriented end portion 812. The proximally oriented portion 810 can comprise a curvature 822 such that a lateral cross section of a distal end 820 of the expandable distal portion 804 is larger than that at a proximal end 818. The lateral cross section can be taken along a plane perpendicular or substantially perpendicular to a longitudinal axis of the sheath shaft 800. For example, a portion of each discrete elongate portion 808 proximal of the curvature 822 can be at an angle relative to the longitudinal axis of the sheath shaft 800. In some instances, a portion of each discrete elongate portion 808 proximal of the curvature 822 can be perpendicular or substantially perpendicular to the longitudinal axis of the sheath shaft 800. In some instances, a portion of each discrete elongate portion 808 distal of the curvature 822 can extend along an axis parallel or substantially parallel to the longitudinal axis of the sheath shaft 800. The proximally oriented portions 810 can be coupled to the proximal portion 802 of the sheath shaft 800. In some instances, each discrete elongate portion 808 can extend distally from the proximal portion 802 along an “L-shaped” path. A portion of each discrete elongate portion 808 proximal of the curvature 822 can form the shorter leg of the “L-shape” and a portion of each discrete elongate portion 808 distal of the curvature 822 forming the longer leg of the “L-shape”. In some instances, the curvature 822 can comprise a step configuration. The plurality of discrete elongate portions 808 can be arranged circumferentially around a delivery catheter conduit 806. The delivery catheter conduit 806 can be configured to slidably receive a catheter shaft 850, such as shown in FIG. 8B. While the expandable distal portion 804 is in the collapsed state, an outer diameter and/or inner diameter at the proximal end 818 of the expandable distal portion 804 can be smaller than that at the distal end 820 of the expandable distal portion 804. An outer diameter and/or inner diameter of the expandable distal portion 804 at distal ends 816 of the plurality discrete elongate portions 808 can be larger than that at proximal ends 814 of the plurality of discrete elongate portions 808.

Referring to FIG. 8B, the expandable distal portion 804 can be in an expanded state while a distal portion 854 of the catheter shaft 850 is received therein. A proximal portion 852 of the catheter shaft 850 can be received in the proximal portion 802 of the sheath shaft 800. For example, a proximal end 856 of the distal portion 854 can be disposed proximate or adjacent to the curvature 822 of the sheath shaft 800. In some instances, the proximal end 856 of the distal portion 854 can abut the curvature 822 and a distal end 858 of the catheter shaft distal portion 854 can be oriented toward the distal ends 816 of the discrete elongate portions 808. In some instances, contact between the proximally oriented end portions 810, including the curvatures 822, of each of the plurality of discrete elongate portions 808 and the catheter shaft distal portion 854 can cause the plurality of discrete elongate portions 808 to rotate outward about a respective proximal end 814 so as to accommodate the catheter shaft distal portion 854.

In some instances, the catheter shaft 850 can be advanced relative to the sheath shaft 800 to deploy the catheter shaft 850. The expandable distal portion 804 can assume the collapsed state when the catheter shaft distal portion 854 is positioned distally of the distal end 820 of the expandable distal portion 804.

In some instances, while in the expanded state, the expandable distal portion 804 described with reference to FIG. 8 can have a lateral cross section at the distal end 820 that is smaller than that at the distal end 720 of the expandable distal portion 704 described with reference to FIG. 7. In some instances, the curvature 822 on the proximally oriented portion 810 can provide the reduced lateral cross-sectional area. The reduced cross-sectional area can facilitate advancement of the sheath shaft 800 through an outer expandable sheath.

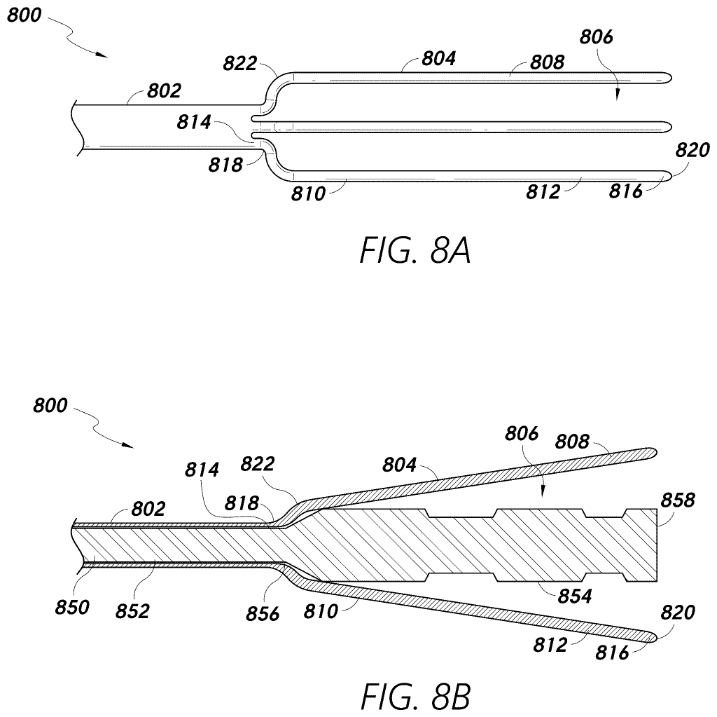

FIG. 9A is a side view of an example of a sheath shaft 900 in a collapsed state, where the sheath shaft 900 comprises a plurality of discrete elongate portions 908 each extending along a convexly curved path. FIG. 9B is a side cross-sectional view of the sheath shaft 900 in an expanded state. Referring to FIG. 9A, each discrete elongate portion 908 can comprise a proximally oriented end portion 910 and a distally oriented end portion 912. A delivery catheter conduit 906 can extend through the catheter shaft 900 and be configured to slidably receive a catheter shaft. The plurality of discrete elongate portions 908 can be circumferentially disposed around the delivery catheter conduit 906. FIG. 9A does not show a catheter shaft within the delivery catheter conduit 906. Each discrete elongate portion 908 can extend along a convexly curved path extending between a corresponding proximally oriented end portion 910 and distally oriented end portion 912. In some instances, each discrete elongate portion 908 can extend along a convexly curved path from a proximal end 914 to a distal end 916. Each of the plurality of discrete elongate portions 908 can be coupled to a proximal portion 902 of the sheath shaft 900, for example extending distally from the proximal portion 902 along a convexly curved path to the distal ends 916 of the respective discrete elongate portions 908.

Referring to FIG. 9B, a catheter shaft 950 is received by the delivery catheter conduit 906. For example, the expandable distal portion 904 can be in an expanded state while a distal portion 954 of a catheter shaft 950 is received therein. A proximal portion 952 of the catheter shaft 950 can be received in the proximal portion 902 of the sheath shaft 900. A proximal end 956 of the distal portion 954 can be disposed proximate or adjacent to the proximal end 918 of the expandable distal portion 904. A distal end 958 of the catheter shaft distal portion 954 can be oriented toward a distal end 920 of the expandable distal portion 904. In some instances, the proximal end 956 of the catheter shaft distal portion 954 can contact the proximally oriented end portions 910 of the plurality of discrete elongate portions 908. Contact between the proximally oriented end portions 910 of each of the plurality of discrete elongate portions 908 and the distal portion 954 of the catheter shaft 950 can push the plurality of discrete elongate portions 908 outward, for example causing a rotation outward about a respective proximal end 914 so as to accommodate the distal portion 954 of the catheter shaft 950. In some instances, the convexly curved discrete elongate portions 908 can facilitate advancement of the sheath shaft 900 through an outer expandable sheath.

In some instances, the catheter shaft 950 can be advanced relative to the sheath shaft 900 to deploy the catheter shaft 950. The expandable distal portion 904 can assume the collapsed state when the catheter shaft distal portion 954 is positioned distally of the distal end 920 of the expandable distal portion 904.

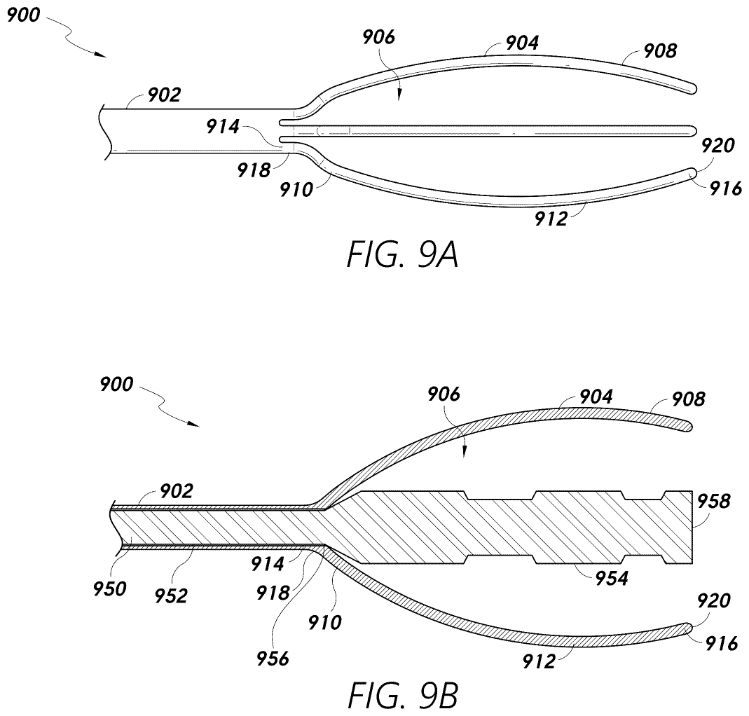

FIG. 10A is a side view, and FIG. 10B is a side cross-sectional view, of another example of a sheath shaft 1000 comprising a plurality of discrete elongate portions 1008 in a collapsed state and in an expanded state, respectively. Referring to FIG. 10A, the sheath shaft 1000 is shown in the collapsed state. Each discrete elongate portion 1008 can comprise a proximally oriented end portion 1010 and a distally oriented end portion 1012. A delivery catheter conduit 1006 can extend through the catheter shaft 1000 and configured to slidably receive a catheter shaft. The plurality of discrete elongate portions 1008 can be circumferentially disposed around the delivery catheter conduit 1006. FIG. 10A does not show a catheter shaft within the delivery catheter conduit 1006. The proximally oriented portion 1010 can comprise a convex curvature 1022 such that a lateral cross section of a distally oriented portion 1012 of the expandable distal portion 1004 is smaller than that at a widest portion of the convex curvature 1022. For example, a lateral cross section at a distal end 1020 of the expandable distal portion 1004 can be smaller than that at a proximal portion of the expandable distal portion 1004, such as at the widest portion of the convex curvature 1022. An outer diameter and/or inner diameter of a proximal portion of the expandable distal portion 1004, such as at the widest portion of the convex curvature 1022, can be larger than that at distal ends 1016 of the plurality discrete elongate portions 1008. In some instances, while the expandable distal portion 1004 is in the collapsed state, the distal end 1016 of each discrete elongate portion 1008 is in contact with at least one distal end 1016 of another discrete elongate portion 1008. In some instances, while the expandable distal portion 1004 is in the collapsed state, distal ends 1016 of the plurality of discrete elongate portions 1008 can be spaced from one another.

Referring to FIG. 10B, the expandable distal portion 1004 can be in an expanded state while a distal portion 1054 of a catheter shaft 1050 is received therein. A proximal portion 1052 of the catheter shaft 1050 can be received in the proximal portion 1002 of the sheath shaft 1000. A proximal end 1056 of the distal portion 1054 can be disposed proximate or adjacent to the proximal end 1018 of the expandable distal portion 1004. A distal end 1058 of the catheter shaft distal portion 1054 can be oriented toward the distal end 1020 of the expandable distal portion 1004. The distal portion 1054 of the catheter shaft 1050 can push against the plurality of discrete elongate portions 1008 such that in the expanded state the plurality of discrete elongate portions 1008 rotates outward about a respective proximal end 1014 so as to accommodate the distal portion 1054 of the catheter shaft 1050. In some instances, the distal portion 1054 of the catheter shaft 1050 can push against the proximally oriented end portions 1010 and/or the distally oriented end portions 1012. In some instances, while in the expanded state, the distally oriented end portions 1012 can be in contact with and be disposed around the catheter shaft distal portion 1054. In some instances, contact between the catheter shaft distal portion 1054 and the distally oriented end portions 1012 while the expandable distal portion 1004 in the expanded state can facilitate advancement of the sheath shaft 1000 through an outer expandable sheath. In some instances, the curvature 1022 can conform or substantially conform to a portion of the catheter shaft distal portion 1054. In some instances, the plurality of elongate portions 1008 can conform or substantially conform to a shape of the catheter shaft distal portion 1054 to facilitate navigation of the expandable distal portion 1004 in the expanded state through an outer sheath shaft.

In some instances, the catheter shaft 1050 can be advanced relative to the sheath shaft 1000 to deploy the catheter shaft 1050. In some instances, the expandable distal portion 1004 can assume an intermediate expandable state when the catheter shaft distal portion 1054 is positioned distally of the distal end 1020 of the expandable distal portion 1004. For example, while the proximal portion 1052 of the catheter shaft 1050 is disposed through the expandable distal portion 1004, the distal end 1016 of each discrete elongate portion 1008 can be in contact with respective portions of the catheter shaft proximal portion 1052.

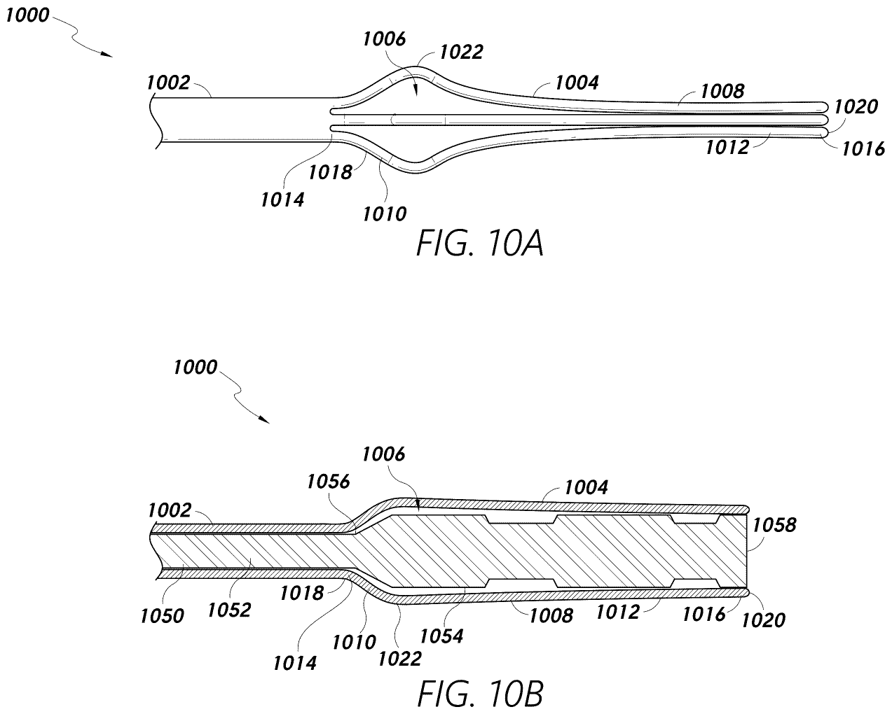

FIG. 11 is a side perspective view of a sheath shaft 1100 comprising three portions each comprising a material different from that of the others. The sheath shaft 1100 can comprise a proximal portion 1102 and an expandable distal portion 1104. While the expandable distal portion 1104 is in an expanded state, the expandable distal portion 1104 can comprise a lateral cross section larger than that of the proximal portion 1102. For example, the expandable distal portion 1104 can be in the expanded state while a catheter shaft is preloaded within a delivery catheter conduit 1106 of the sheath shaft 1100. FIG. 11 shows the expandable distal portion 1104 in the expanded state. The proximal portion 1102 can comprise a first portion 1108 and a second portion 1110, the first portion 1108 being more proximal than the second portion 1110. The first portion 1108 can comprise a material more rigid than that of the second portion 1110. In some instances, the first portion 1108 can comprise a rigid portion and the second portion 1110 can comprise a flexible portion. In some instances, the rigidity of the first portion 1108 can facilitate desired torqueability of the sheath shaft 1100. A combination of the improved torqueability of the first portion 1108 and the flexibility of the second portion 1110 can facilitate navigation of the sheath shaft 1100 through tortuous anatomical pathways.

The first portion 1108 can comprise any number of materials to provide the desired rigidity. In some instances, the first portion 1108 can be braid reinforced. The first portion can comprise a braid reinforced tubular configuration. The second portion 1110 can comprise any number of materials to provide the desired flexibility. In some instances, the second portion 1110 can be braid and/or coil reinforced. In some instances, the second portion 1110 can comprise a braid and/or coil reinforced tubular configuration.

In some instances, the expandable distal portion 1104 can be coupled to the second portion 1110 of the proximal portion 1102. The expandable distal portion 1104 can be both an expandable and flexible portion. In some instances, the expandable distal portion 1104 can extend distally from the second portion 1110. For example, the expandable and flexible portion can extend distally from a flexible portion. A proximal end 1120 of the expandable distal portion 1104 can be adjacent to a distal end 1116 of the second portion 1110. A proximal end 1114 of the second portion 1110 can be adjacent to a distal end 1112 of the first portion 1108. The expandable distal portion 1104 can have one or more features as described herein. In some instances, the expandable distal portion 1104 can be coil reinforced. For example, the expandable distal portion 1104 can comprise an expandable coil 1118 extending from the proximal end 1120 to a distal end 1122.

Alternatively, the sheath shaft 1100 can comprise a distal portion that is not expandable. For example, the sheath shaft 1100 can comprise a non-expandable distal portion that has a larger lateral cross section, such as compared to the proximal portion 1102 of the sheath shaft 1100. The non-expandable distal portion can have the same configuration as that of the expandable distal portion 1104 in the expanded state. The non-expandable distal portion can comprise a flexible material. In some instances, the non-expandable distal portion can be a flexible portion comprising a size, including a cross-sectional size, and shape as that of the expandable distal portion 1104 in the expanded state.

The sheath shafts 400, 500, 600, 700, 800, 900, 1000, 1100 described with reference to FIGS. 4, 5, 6, 7, 8, 9, 10 and 11 can have one or more other features of the sheath shaft 230 described with reference to FIG. 2. In some instances, the sheath shafts 400, 500, 600, 700, 800, 900, 1000, 1100 described with reference to FIGS. 4, 5, 6, 7, 8, 9, 10 and 11 can be a part of one or more medical device delivery systems described herein, including the medical device delivery system 100 described with reference to FIG. 2.

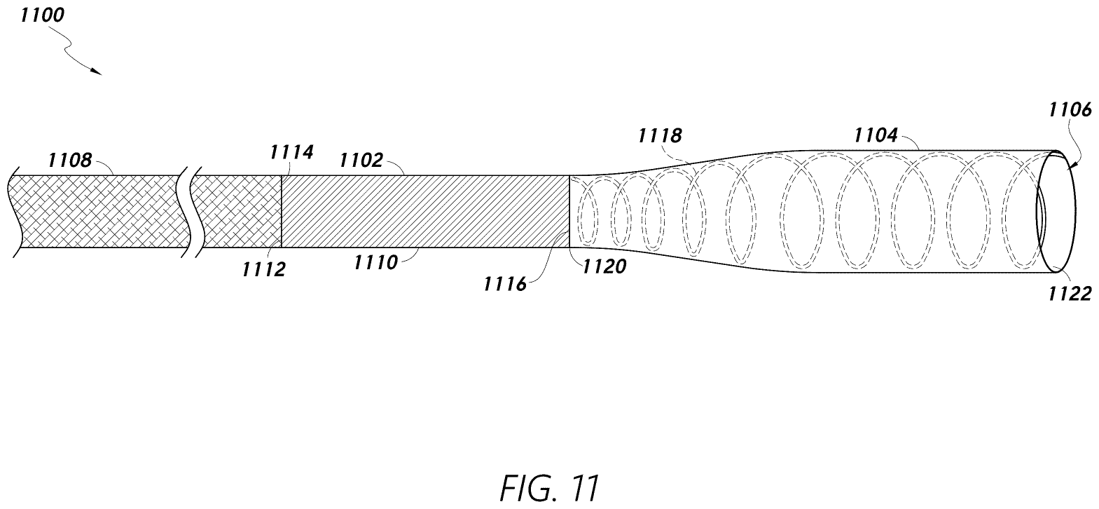

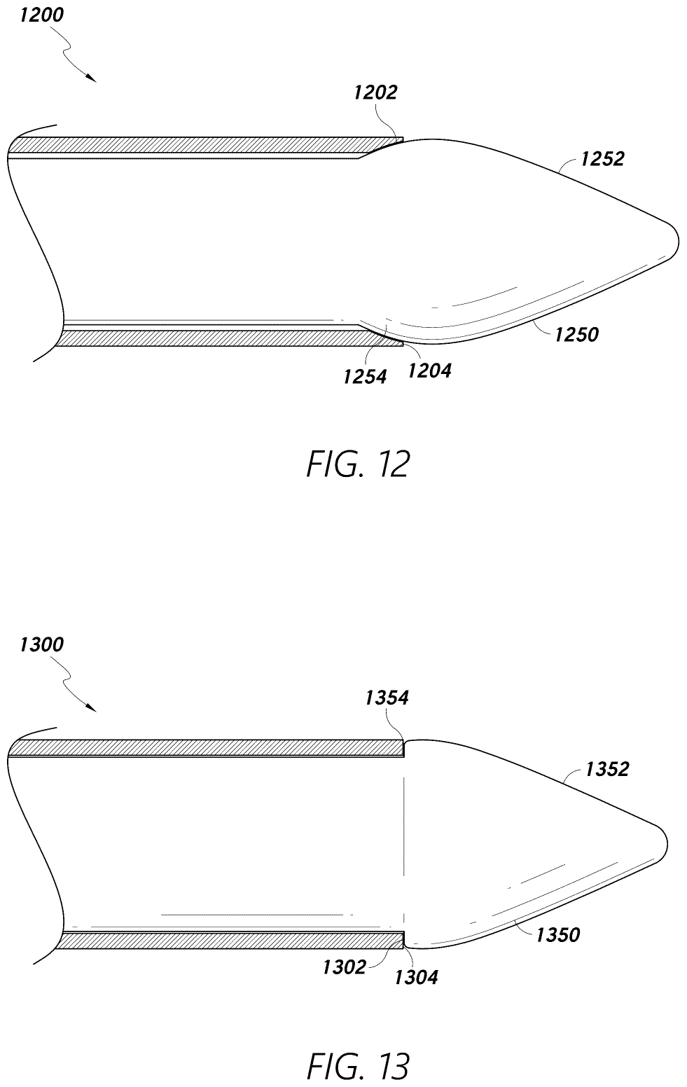

FIGS. 12 and 13 are side cross-sectional views showing distal ends of examples of expandable distal portions, where the distal ends can be configured to mate with a distal portion of a medical device received through the respective expandable distal portion. FIG. 12 shows an expandable distal portion 1200 comprising a circumferential tapered portion 1202 at a distal end 1204. The expandable distal portion 1200 can be a part of an in-line sheath comprising one or more other features as described herein. The remainder of the in-line sheath is not shown for simplicity. Referring to FIG. 12, the circumferential tapered portion 1202 can extend circumferentially around the distal end 1204 of the expandable distal portion 1200. The circumferential tapered portion 1202 can have a slope and/or curvature oriented toward a longitudinal axis of the expandable distal portion 1200. The circumferential tapered portion 1202 can be configured to engage with a corresponding portion of a medical device 1250 while the medical device 1250 is preloaded within the in-line sheath. For example, while the medical device 1250 is preloaded within the in-line sheath, the medical device 1250 can comprise a distal portion 1252 disposed distally of the distal end 1204 of the expandable distal portion 1200. The medical device 1250 can be preloaded within a delivery catheter preloaded within the in-line sheath such that the distal portion 1252 of the medical device 1250 is distal of a distal end of the delivery catheter, such as a distal end of the catheter shaft of the delivery catheter. The delivery catheter is not shown for simplicity.

The circumferential tapered portion 1202 can be seated against a corresponding tapered portion 1254 of the medical device 1250. The corresponding tapered portion 1254 can have a same or similar configuration as that of the circumferential tapered portion 1202, including for example a same or similar slope and/or curvature. In some instances, the corresponding tapered portion 1254 can be on a proximally oriented surface of the distal portion 1252 such that the corresponding tapered portion 1254 can mate with the circumferential tapered portion 1202 while the medical device 1250 is preloaded within the in-line sheath. In some instances, the corresponding tapered portion 1254 can be a curved portion such that the circumferential tapered portion 1202 can be configured to mate with a curved portion of the medical device 1250 disposed distally of the expandable distal portion 1200.

Referring to FIG. 13, an expandable distal portion 1300 which has a distal end 1304 comprising a circumferential lateral portion 1302 configured to engage with a corresponding portion of a medical device 1350. The expandable distal portion 1300 can be a part of an in-line sheath comprising one or more other features as described herein. The remainder of the in-line sheath is not shown for simplicity. The circumferential lateral portion 1302 can extend circumferentially around the distal end 1304 of the expandable distal portion 1300. The circumferential lateral portion 1302 can comprise a surface perpendicular or substantially perpendicular to a longitudinal axis of the expandable distal portion 1300. The circumferential lateral portion 1302 be configured to engage with a corresponding lateral portion 1354 of the medical device 1350 disposed distally of the expandable distal portion 1300. For example, a distal portion 1352 of the medical device 1350 can be disposed distally of the distal end 1304 of the expandable distal portion 1300. In some instances, the circumferential lateral portion 1302 can comprise a proximally oriented portion of the distal portion 1352 of the medical device 1350 that is perpendicular or substantially perpendicular to the longitudinal axis of the expandable distal portion 1300. For example, a proximally oriented flat portion of the medical device 1350 disposed distally of the distal end 1304 of the expandable distal portion 1300 can be seated against the circumferential lateral portion 1302.

Mating between the distal portion of the medical device and the distal end of the sheath shaft can facilitate secure positioning of the medical device within the delivery catheter and/or in-line sheath during advancement of the medical delivery system through an outer expandable sheath. The expandable distal portions 1200, 1300 described with reference to FIGS. 12 and 13 can be a part of one or more sheath shafts described herein, including for example the sheath shafts 400, 500, and 1100 described with reference to FIGS. 4, 5 and 11.

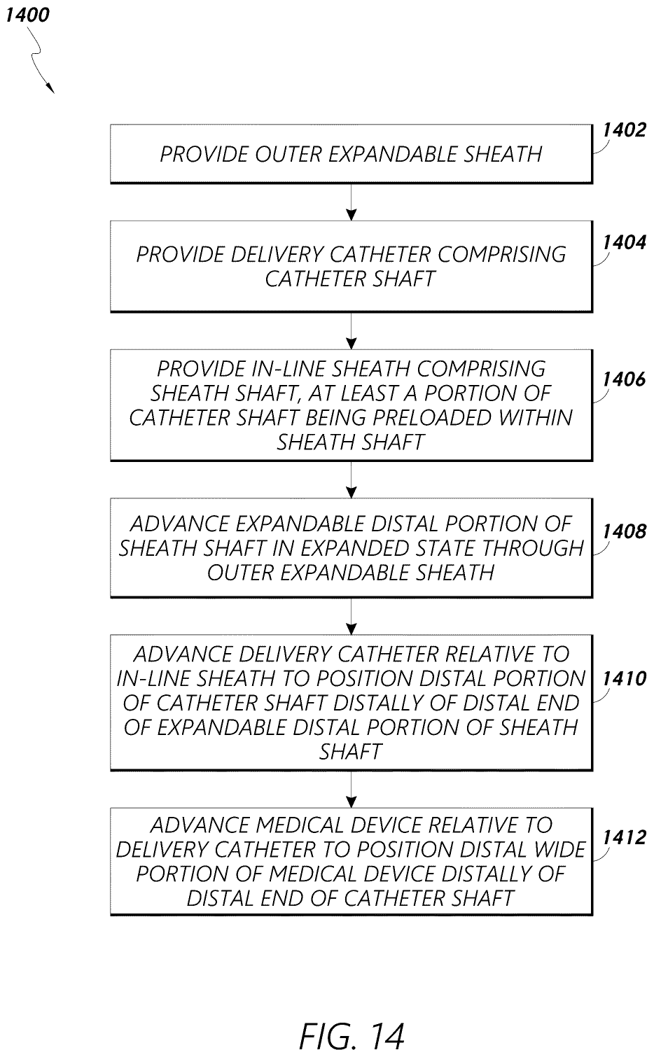

FIG. 14 is a process flow diagram of an example of a process 1400 for delivering a medical device using one or more of the medical delivery systems as described herein. In block 1402, the process can involve providing an outer expandable sheath. The outer expandable sheath can comprise an outer sheath hub and an outer sheath shaft. In some instances, the outer expandable sheath can be positioned into an anatomical pathway to facilitate subsequent positioning of a medical device through the outer expandable sheath. In some instances, the outer expandable sheath can be an introducer access sheath. The outer sheath shaft can comprise at least a portion of which is expandable, and configured to be in a collapsed state prior to the insertion of the medical device delivery system through the outer sheath shaft, and an expanded state to accommodate the medical device delivery system while the medical device delivery system is positioned through the outer sheath shaft.

In block 1404, the process can involve providing a delivery catheter. A medical device can be preloaded within the delivery catheter. The delivery catheter can comprise a catheter shaft that comprises a proximal portion slidably receiving a proximal narrow portion of the medical device. A distal portion of the catheter shaft can be slidably receiving a distal wide portion of the medical device. The distal wide portion of the medical device can have a lateral cross-sectional area larger than that of the proximal narrow portion of the medical device. For example, an inner and/or outer diameter of the distal wide portion can be larger than that of the proximal narrow portion. In some instances, the distal portion of the catheter shaft can have a larger lateral cross-sectional area than that of the proximal portion of the catheter shaft to receive corresponding portions of the medical device. For example, an inner and/or outer diameter of the distal portion of the catheter shaft can be larger than that of the proximal portion of the catheter shaft.

In block 1406, the process can involve providing an in-line sheath comprising a sheath shaft, at least a portion of the catheter shaft being preloaded within the sheath shaft. The sheath shaft can comprise a proximal portion slidably receiving the proximal portion of the catheter shaft. An expandable distal portion of the sheath shaft, in an expanded state, can be slidably receiving the distal portion of the catheter shaft.

In block 1408, the process can involve advancing the expandable distal portion of the sheath shaft in the expanded state through the outer expandable sheath. The delivery catheter can be preloaded within the in-line sheath such that at least a portion of the catheter shaft is carried by the sheath shaft as the expandable distal portion of the sheath shaft is advanced through the outer expandable sheath. For example, at least a portion of the sheath shaft, including the expandable distal portion, can be advanced through the outer sheath hub and the outer sheath shaft. The in-line sheath can be translated distally relative to the outer expandable sheath until the expandable distal portion is advanced through an opening at a distal end of the outer sheath shaft and disposed distally of the distal end of the outer sheath shaft.

The sheath shaft can protect the catheter shaft, and the medical device preloaded therein, as the sheath shaft is translated distally relative to the outer expandable sheath and advanced through the outer sheath hub and outer sheath shaft. For example, the outer sheath hub can comprise one or more seals, including hemostatic seals. The sheath shaft can provide column strength along a longitudinal dimension of the sheath shaft as the sheath shaft is advanced through the one or more seals. The sheath shaft can protect the catheter sheath and medical device against interaction with the one or more seals of the outer sheath hub. As described herein, the outer sheath shaft can comprise at least a portion in a collapsed state prior to insertion of the delivery system. The sheath shaft can provide column strength and/or protection for the catheter sheath and medical device as the sheath shaft is advanced through the collapsed outer sheath shaft.

In block 1410, the process can involve advancing the delivery catheter relative to the in-line sheath to position the distal portion of the catheter shaft distally of a distal end of the expandable distal portion of the sheath shaft. For example, the delivery catheter can be translated distally relative to the in-line sheath to advance the distal portion of the catheter shaft through a distal opening on a distal end of the sheath shaft to facilitate deployment of the medical device. The distal portion of the catheter shaft can be positioned through the distal opening on the distal end of the sheath shaft after the expandable distal portion of the sheath shaft is positioned distally of the distal of the outer sheath shaft. In some instances, the expandable distal portion of the sheath shaft can assume a collapsed state after the distal portion of the catheter shaft is advanced out of the expandable distal portion, such as while a portion of the proximal portion of the catheter shaft is received through the expandable distal portion. As described herein, the proximal portion of the catheter shaft can have a smaller lateral cross-sectional size as compared to the distal portion of the catheter shaft.

In block 1412, the process can involve advancing the medical device relative to the delivery catheter to position the distal wide portion of the medical device distally of a distal end of the catheter shaft. In some instances, the medical device can be deployed from the delivery catheter after the distal portion of the catheter shaft is positioned distally of the distal end of the sheath shaft.

In some instances, the outer expandable sheath can be inserted through an opening in an iliac artery or a femoral artery. The in-line sheath comprising the delivery catheter and medical device preloaded therein can be advanced through the outer expandable sheath, corresponding portions of the outer expandable sheath assuming an expanded state as the in-line sheath is positioned through the outer expandable sheath. The expandable distal portion of the sheath shaft can be in the expanded state as it is advanced through the outer expandable sheath. The expandable distal portion of the sheath shaft can be positioned distally of a distal end of the outer expandable sheath and into a target anatomy, such as an aorta, including a descending aorta or an abdominal aorta. After the expandable distal portion of the sheath shaft is advanced out of the outer expandable sheath, the outer expandable sheath can assume a collapsed state, facilitating blood flow around the outer expandable sheath. The outer expandable sheath in the collapsed state can facilitate blood flow proximate and/or adjacent to the insertion site, for example a region having narrower blood vessels, such as the iliac artery or the femoral artery.

After the expandable distal portion of the sheath shaft is at its target position, the delivery catheter and/or the medical device can then be advanced to a target site. The delivery catheter can be translated distally relative to the in-line sheath to deploy the distal portion of the catheter sheath from the expandable distal portion of the sheath shaft. Subsequently, the distal wide portion of the medical device can be deployed from the delivery catheter. As described herein, the expandable distal portion of the sheath shaft can be positioned into a larger blood vessel which can more easily accommodate the expandable distal portion. After the distal portion of the catheter sheath carrying the distal wide portion of the medical device therein is advanced out of a distal end of the expandable distal portion, the expandable distal portion of the sheath shaft can assume a collapsed state. The collapsed expandable distal portion can facilitate blood flow therearound, such as within the descending aorta or abdominal aorta.

The medical device can be used to treat any number of conditions. In some instances, the medical device can be configured to treat a heart condition. In some instances, the distal wide portion of the medical device can be advanced into a target site in a heart chamber, including a right ventricle or a left ventricle.

In some instances, a catheter engagement feature of the sheath hub can be activated to provide frictional contact between the in-line sheath and the delivery catheter, such as to prevent or reduce proximal and distal translation of the delivery catheter relative to the in-line sheath. For example, after the distal portion of the catheter shaft is advanced out of the distal end of the sheath shaft and positioned at a desired location, the relative position of the delivery catheter and the in-line sheath can be fixed. Frictional contact between the engagement feature of the sheath hub and the delivery catheter can prevent or reduce translational and/or rotational movement of the delivery catheter relative to the in-line sheath. In some instances, the medical device can be advanced out of the distal end of the catheter shaft after the relative position of the delivery catheter and the in-line sheath is secured.

In some instances, an outer sheath engagement feature of the sheath hub configured to engage with a hub of the outer expandable sheath can be activated to prevent or reduce distal and proximal translation of the in-line sheath relative to the outer expandable sheath. For example, after the expandable distal portion of the sheath shaft is advanced out of the distal end of the outer sheath shaft and positioned at a desired location, the relative position of the in-line sheath and the outer expandable sheath can be fixed. In some instances, the distal portion of the catheter shaft can be advanced out of the distal end of the sheath shaft after the relative position of the outer expandable sheath and the in-line sheath is secured.

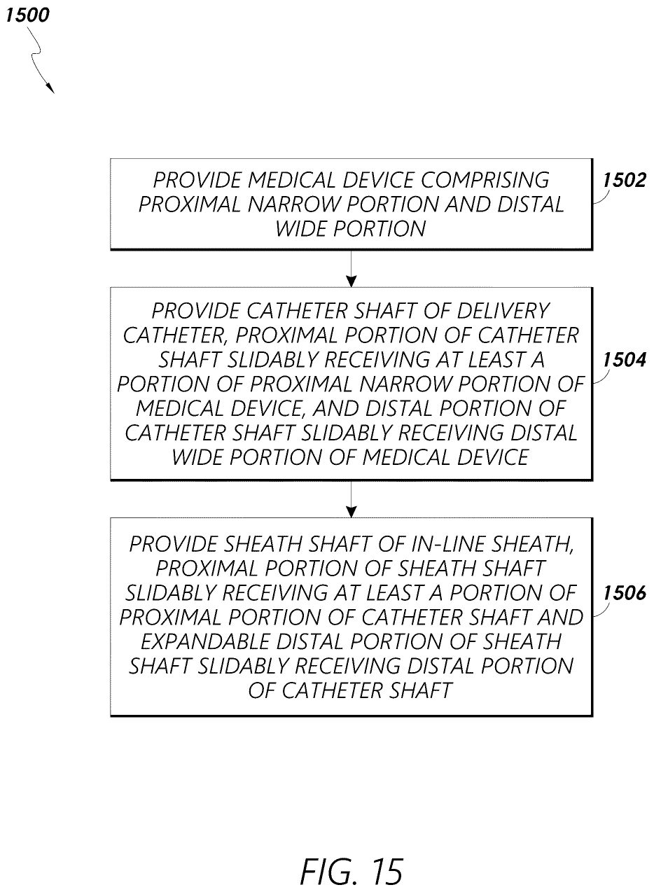

FIG. 15 is a process flow diagram showing a manufacturing process 1500 for manufacturing one or more medical device delivery systems as described herein. In block 1502, the process can involve providing a medical device comprising a proximal narrow portion and a distal wide portion. In block 1504, the process can involve providing a catheter shaft of a delivery catheter. The catheter shaft of the delivery catheter can be provided around at least a portion of the medical device such that a proximal portion of the catheter shaft is slidably receiving at least a portion of the proximal narrow portion of the medical device. A distal portion of the catheter shaft can be slidably receiving the distal wide portion of the medical device. In block 1506, the process can involve providing a sheath shaft of an in-line sheath. The sheath shaft can be around at least a portion of the catheter shaft. A proximal portion of the sheath shaft can be slidably receiving at least a portion of the proximal portion of the catheter shaft and an expandable distal portion of the sheath shaft, in an expanded state, slidably receiving the distal portion of the catheter shaft. As described herein, in some instances, the expandable distal portion can comprise a tubular configuration, including a cylindrical configuration. Alternatively or in combination, the expandable distal portion can comprise a plurality of discrete elongate portions. The plurality of discrete elongate portions can be circumferentially disposed around the distal portion of the catheter shaft.

In some instances, the in-line sheath and the delivery catheter can be assembled together. In some instances, a catheter hub can be coupled to the catheter shaft, and/or a sheath hub can be coupled to the sheath shaft, after the sheath shaft is provided around at least a portion of the catheter shaft. For example, the catheter hub can be coupled to the proximal portion of the catheter sheath and/or the sheath hub can be coupled to the proximal portion of the sheath shaft, after the after the sheath shaft is positioned around at least a portion of the catheter shaft.

### ADDITIONAL DESCRIPTION OF EXAMPLES

Provided below is a list of examples, each of which may include aspects of any of the other examples disclosed herein. Furthermore, aspects of any example described above may be implemented in any of the numbered examples provided below.

### Example 1

A medical device delivery system comprising a delivery catheter and an in-line sheath. The delivery catheter can comprise a catheter hub, and a catheter shaft. The catheter shaft can include a medical device conduit extending through the catheter shaft and configured to slidably receive at least a portion of a medical device, wherein a proximal portion of the catheter shaft extends distally from the catheter hub and is configured to slidably receive a proximal narrow portion of the medical device, and wherein a distal portion of the catheter shaft is configured to slidably receive a distal wide portion of the medical device, and wherein the distal wide portion of the medical device has a diameter wider than that of the proximal narrow portion of the medical device. The delivery catheter can be preloaded within the in-line sheath. The in-line sheath can comprise a sheath hub, and a sheath shaft including a delivery catheter conduit extending through the sheath shaft and configured to slidably receive at least a portion of the catheter shaft. A proximal portion of the sheath shaft can extend distally from the sheath hub and slidably receive at least a portion of the proximal portion of the catheter shaft, and an expandable distal portion of the sheath shaft can be configured to assume an expanded state or a collapsed state, and wherein the expandable distal portion is configured to slidably receive the distal portion of the catheter shaft when in its expanded state.

### Example 2

The system of any example herein, in particular example 1, wherein the expandable distal portion of the sheath shaft comprises a tubular configuration.

### Example 3

The system of any example herein, in particular example 2, wherein the expandable distal portion, while in the expanded state, comprises a cylindrical shape.

### Example 4

The system of any example herein, in particular example 2 or 3, wherein an expandable coil extends along a longitudinal portion of the expandable distal portion, coils of the expandable coil extending around a corresponding portion of the delivery catheter conduit.

### Example 5

The system of any example herein, in particular examples 2 to 4, wherein the expandable distal portion comprises a shape memory material.

### Example 6

The system of any example herein, in particular examples 2 to 5, wherein a distal end of the expandable distal portion comprises a circumferential tapered portion having a sloped surface oriented toward a longitudinal axis of the sheath shaft, the circumferential tapered portion being configured to engage with a curved portion of the medical device disposed distally of the expandable distal portion.

### Example 7

The system of any example herein, in particular examples 2 to 5, wherein a distal end of the expandable distal portion comprises a circumferential lateral portion perpendicular to a longitudinal axis of the sheath shaft, the circumferential lateral portion being configured to engage with a flat portion of the medical device disposed distally of the expandable distal portion.

### Example 8

The system of any example herein, in particular example 1, wherein the expandable distal portion comprises a plurality of discrete elongate portions circumferentially disposed around the delivery catheter conduit and partially defining a corresponding portion of the delivery catheter conduit, each discrete elongate portion comprising a proximally oriented end portion and a distally oriented end portion, the proximally oriented portions being coupled to one another and the distally oriented end portions being spaced from one another, wherein, while the expandable distal portion is in the expanded state, the distally oriented end portions are configured to be disposed further apart from one another than while in the expandable distal portion is in the collapsed state.

### Example 9

The system of any example herein, in particular example 8, wherein the expandable distal portion comprises three discrete elongate portions.

### Example 10

The system of any example herein, in particular example 8 or 9, wherein, while the expandable distal portion is in the collapsed state, each discrete elongate portion extends distally along an axis parallel to a longitudinal axis of the sheath shaft to provide an expandable distal portion comprising a uniform outer diameter along a longitudinal dimension of the expandable distal portion.

### Example 11

The system of any example herein, in particular example 8 or 9, wherein each discrete elongate portion extends along a convexly curved path from a corresponding proximal end to a corresponding distal end.

### Example 12

The system of any example herein, in particular example 8 or 9, wherein, while the expandable distal portion is in the collapsed state, a proximally oriented portion of each discrete elongate portion comprises a curvature to provide an outer diameter of the expandable distal portion at distal ends of the plurality of discrete elongate portions that is larger than that at proximal ends of the plurality of discrete elongate portions.

### Example 13

The system of any example herein, in particular example 8 or 9, wherein a proximally oriented portion of each discrete elongate portion comprises a convex curvature to provide a proximal portion of the expandable distal portion comprising an outer diameter wider than that of a distal portion of the expandable distal portion.

### Example 14

The system of any example herein, in particular example 13, wherein, while the expandable distal portion is in the collapsed state, a distal end of each discrete elongate portion is in contact with at least one other discrete elongate portion.

### Example 15

The system of any example herein, in particular example 13, wherein, while the expandable distal portion is in the collapsed state, distal ends of the plurality of discrete elongate portions are spaced from one another.

### Example 16

The system of any example herein, in particular examples 1 to 15, wherein the proximal portion of the sheath shaft comprises a rigid portion and a flexible portion, the rigid portion being proximal of the flexible portion.

### Example 17

The system of any example herein, in particular example 16, wherein the expandable distal portion extends distally from the flexible portion.

### Example 18

The system of any example herein, in particular examples 1 to 17, wherein the sheath hub comprises a catheter engagement feature configured to engage with a portion of the delivery catheter to prevent distal and proximal translation of the delivery catheter relative to the in-line sheath.

### Example 19

The system of any example herein, in particular example 18, wherein the catheter engagement feature comprises a catheter shaft engagement component configured to frictionally contact a portion of the catheter shaft.

### Example 20

The system of any example herein, in particular examples 1 to 19, wherein the expandable distal portion of the sheath shaft and at least a portion of the proximal portion of the sheath shaft are configured to be advanced through an outer expandable sheath; and the sheath hub comprises an outer sheath engagement feature configured to engage with the outer expandable sheath to prevent distal and proximal translation of the in-line sheath relative to the outer expandable sheath

### Example 21

The system of any example herein, in particular example 20, wherein the outer sheath engagement feature comprises a hub engagement component configured to engage with a hub of the outer expandable sheath.

### Example 22

A method of delivering a medical device, the method comprising providing an outer expandable sheath; providing a delivery catheter comprising a catheter shaft having a proximal portion slidably receiving a proximal narrow portion of the medical device and a distal portion slidably receiving a distal wide portion of a medical device; and providing an in-line sheath comprising a sheath shaft, at least a portion of the catheter shaft being preloaded within the sheath shaft. The sheath shaft can comprise a proximal portion slidably receiving the proximal portion of the catheter shaft, and an expandable distal portion slidably receiving, in an expanded state, the distal portion of the catheter shaft. The method can include advancing the expandable distal portion of the sheath shaft in the expanded state through the outer expandable sheath; advancing the delivery catheter relative to the in-line sheath to position the distal portion of catheter shaft distally of a distal end of the expandable distal portion of the sheath shaft; and advancing the medical device relative to the delivery catheter to position the distal wide portion of the medical device distally of a distal end of the catheter shaft.

### Example 23

The method of any example herein, in particular example 22, and further comprising advancing the distal wide portion of the medical device into a heart chamber.

### Example 24

The method of any example herein, in particular example 22 or 23, and further comprising activating a catheter engagement feature of a sheath hub of the in-line sheath to provide frictional contact between the in-line sheath and the delivery catheter to prevent proximal and distal translation of the delivery catheter relative to the in-line sheath.

### Example 25

The method of any example herein, in particular examples 22 to 24, and further comprising activating an outer sheath engagement feature of a sheath hub of the in-line sheath to engage with a hub of the outer expandable sheath to prevent distal and proximal translation of the in-line sheath relative to the outer expandable sheath.

The above method(s) can be performed on a living animal or on a simulation, such as on a cadaver, cadaver heart, anthropomorphic ghost, simulator (e.g., with body parts, heart, tissue, etc. being simulated).

### Example 26

A method of manufacturing a medical device delivery system, the method comprising providing a medical device comprising a proximal narrow portion and a distal wide portion; providing a catheter shaft of a delivery catheter, a proximal portion of the catheter shaft slidably receiving at least a portion of the proximal narrow portion of the medical device, and a distal portion of the catheter shaft slidably receiving the distal wide portion of the medical device; and providing a sheath shaft of an in-line sheath, a proximal portion of the sheath shaft slidably receiving at least a portion of the proximal portion of the catheter shaft and an expandable distal portion of the sheath shaft, in an expanded state, slidably receiving the distal portion of the catheter shaft.

### Example 27

The method of any example herein, in particular example 26, and further comprising providing a catheter hub and coupling the catheter hub to the proximal portion of the catheter sheath.

### Example 28

The method of any example herein, in particular example 26 or 27, and further comprising providing a sheath hub and coupling the sheath hub to the proximal portion of the sheath shaft.

### Example 29

The method of any example herein, in particular examples 26 to 28, wherein providing the sheath shaft of an in-line sheath around at least a portion of the catheter shaft comprises providing an expandable distal portion comprising a tubular configuration.

### Example 30

The method of any example herein, in particular examples 26 to 28, wherein providing the sheath shaft of an in-line sheath around at least a portion of the catheter shaft comprises providing an expandable distal portion comprising a plurality of discrete elongate portions circumferentially disposed around the distal portion of the catheter shaft.

The above method(s) can be performed on a living animal or on a simulation, such as on a cadaver, cadaver heart, anthropomorphic ghost, simulator (e.g., with body parts, heart, tissue, etc. being simulated).

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which the examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A medical device delivery system, comprising:
a delivery catheter comprising:
a catheter hub; and
a catheter shaft including a medical device conduit extending through the catheter shaft and configured to slidably receive at least a portion of a medical device,
wherein a proximal portion of the catheter shaft extends distally from the catheter hub and is configured to slidably receive a proximal narrow portion of the medical device, and
wherein a distal portion of the catheter shaft is configured to slidably receive a distal wide portion of the medical device, and wherein the distal wide portion of the medical device has a diameter wider than that of the proximal narrow portion of the medical device; and
an in-line sheath, the delivery catheter being preloaded within the in-line sheath, the in-line sheath comprising:
a sheath hub; and
a sheath shaft including a delivery catheter conduit extending through the sheath shaft and configured to slidably receive at least a portion of the catheter shaft, wherein a proximal portion of the sheath shaft extends distally from the sheath hub and is configured to slidably receive at least a portion of the proximal portion of the catheter shaft, and wherein an expandable distal portion of the sheath shaft is configured to assume an expanded state or a collapsed state, and wherein the expandable distal portion of the sheath shaft is configured to slidably receive the distal portion of the catheter shaft when in its expanded state.

2. The system of claim 1, wherein the expandable distal portion of the sheath shaft comprises a tubular configuration.

3. The system of claim 2, wherein the expandable distal portion, while in the expanded state, comprises a cylindrical shape.

4. The system of claim 2, wherein an expandable coil extends along a longitudinal portion of the expandable distal portion, coils of the expandable coil extending around a corresponding portion of the delivery catheter conduit.

5. The system of claim 2, wherein the expandable distal portion comprises a shape memory material.

6. The system of claim 2, wherein a distal end of the expandable distal portion comprises a circumferential tapered portion having a sloped surface oriented toward a longitudinal axis of the sheath shaft, the circumferential tapered portion being configured to engage with a curved portion of the medical device disposed distally of the expandable distal portion.

7. The system of claim 2, wherein a distal end of the expandable distal portion comprises a circumferential lateral portion perpendicular to a longitudinal axis of the sheath shaft, the circumferential lateral portion being configured to engage with a flat portion of the medical device disposed distally of the expandable distal portion.

8. The system of claim 1, wherein the expandable distal portion comprises:
a plurality of discrete elongate portions circumferentially disposed around the delivery catheter conduit and partially defining a corresponding portion of the delivery catheter conduit, each discrete elongate portion comprising a proximally oriented end portion and a distally oriented end portion, the proximally oriented portions being coupled to one another and the distally oriented end portions being spaced from one another,
wherein, while the expandable distal portion is in the expanded state, the distally oriented end portions are configured to be disposed further apart from one another than while in the expandable distal portion is in the collapsed state.

9. The system of claim 8, wherein, while the expandable distal portion is in the collapsed state, each discrete elongate portion extends distally along an axis parallel to a longitudinal axis of the sheath shaft to provide an expandable distal portion comprising a uniform outer diameter along a longitudinal dimension of the expandable distal portion.

10. The system of claim 8, wherein each discrete elongate portion extends along a convexly curved path from a corresponding proximal end to a corresponding distal end.

11. The system of claim 8, wherein, while the expandable distal portion is in the collapsed state, a proximally oriented portion of each discrete elongate portion comprises a curvature to provide an outer diameter of the expandable distal portion at distal ends of the plurality of discrete elongate portions that is larger than that at proximal ends of the plurality of discrete elongate portions.

12. The system of claim 8, wherein a proximally oriented portion of each discrete elongate portion comprises a convex curvature to provide a proximal portion of the expandable distal portion comprising an outer diameter wider than that of a distal portion of the expandable distal portion.

13. The system of claim 12, wherein, while the expandable distal portion is in the collapsed state, a distal end of each discrete elongate portion is in contact with at least one other discrete elongate portion or distal ends of the plurality of discrete elongate portions are spaced from one another.

14. The system of claim 2, wherein the proximal portion of the sheath shaft comprises a rigid portion and a flexible portion, the rigid portion being proximal of the flexible portion, and wherein the expandable distal portion extends distally from the flexible portion.

15. The system of claim 2, wherein the sheath hub comprises a catheter engagement feature configured to frictionally contact with a portion of the delivery catheter to prevent distal and proximal translation of the delivery catheter relative to the in-line sheath.

16. The system of claim 2, wherein:
the expandable distal portion of the sheath shaft and at least a portion of the proximal portion of the sheath shaft are configured to be advanced through an outer expandable sheath; and
the sheath hub comprises an outer sheath engagement feature configured to engage with a hub of the outer expandable sheath to prevent distal and proximal translation of the in-line sheath relative to the outer expandable sheath.

17. A method of delivering a medical device, the method comprising:
providing an outer expandable sheath;
providing a delivery catheter comprising a catheter shaft having a proximal portion slidably receiving a proximal narrow portion of the medical device and a distal portion slidably receiving a distal wide portion of a medical device;
providing an in-line sheath comprising a sheath shaft, at least a portion of the catheter shaft being preloaded within the sheath shaft, the sheath shaft comprising:
a proximal portion slidably receiving the proximal portion of the catheter shaft; and
an expandable distal portion slidably receiving, in an expanded state, the distal portion of the catheter shaft;
advancing the expandable distal portion of the sheath shaft in the expanded state through the outer expandable sheath;
advancing the delivery catheter relative to the in-line sheath to position the distal portion of catheter shaft distally of a distal end of the expandable distal portion of the sheath shaft; and
advancing the medical device relative to the delivery catheter to position the distal wide portion of the medical device distally of a distal end of the catheter shaft.

18. The method of claim 17, and further comprising advancing the distal wide portion of the medical device into a heart chamber.

19. The method of claim 17, and further comprising activating a catheter engagement feature of a sheath hub of the in-line sheath to provide frictional contact between the in-line sheath and the delivery catheter to prevent proximal and distal translation of the delivery catheter relative to the in-line sheath.

20. The method of claim 17, and further comprising activating an outer sheath engagement feature of a sheath hub of the in-line sheath to engage with a hub of the outer expandable sheath to prevent distal and proximal translation of the in-line sheath relative to the outer expandable sheath.
